SEQUENCE LISTING (198 sequences)

SEQ ID NO: 1 - DNA, 43 bp, Artificial Sequence
  Sense and antisense primer for the genome section 39.394.075 - 39.569.394 on chromosome 17
tgtaaaacgacggccagtcttagtttcaccagtactcaatccc

SEQ ID NO: 2 - DNA, 43 bp, Artificial Sequence
  Sense and antisense primer for the genome section 39.394.075 - 39.569.394 on chromosome 17
caggaaacagctatgaccctcagtctcagaatacaccgttaag

SEQ ID NO: 3 - DNA, 43 bp, Artificial Sequence
  Sense and antisense primer for the genome section 39.394.075 - 39.569.394 on chromosome 17
tgtaaaacgacggccagtctagggttacaggttgcacataata

SEQ ID NO: 4 - DNA, 43 bp, Artificial Sequence
  Sense and antisense primer for the genome section 39.394.075 - 39.569.394 on chromosome 17
caggaaacagctatgaccgccttcattttaagtgccataactg

SEQ ID NO: 5 - DNA, 43 bp, Artificial Sequence
  Sense and antisense primer for the genome section 39.394.075 - 39.569.394 on chromosome 17
tgtaaaacgacggccagttggcctccagttacaacatataaaa

SEQ ID NO: 6 - DNA, 43 bp, Artificial Sequence
  Sense and antisense primer for the genome section 39.394.075 - 39.569.394 on chromosome 17
caggaaacagctatgaccctctgtaggggtggcatactataat

SEQ ID NO: 7 - DNA, 42 bp, Artificial Sequence
  Sense and antisense primer for the genome section 39.394.075 - 39.569.394 on chromosome 17
tgtaaaacgacggccagtcccgactcaaatcttcagtatctg

SEQ ID NO: 8 - DNA, 42 bp, Artificial Sequence
  Sense and antisense primer for the genome section 39.394.075 - 39.569.394 on chromosome 17
caggaaacagctatgaccagggtgtgaaaacttttaagggtt

SEQ ID NO: 9 - DNA, 43 bp, Artificial Sequence
  Sense and antisense primer for the genome section 39.394.075 - 39.569.394 on chromosome 17
tgtaaaacgacggccagtaaagaccaggtgacatttactcttc

SEQ ID NO: 10 - DNA, 43 bp, Artificial Sequence
  Sense and antisense primer for the genome section 39.394.075 - 39.569.394 on chromosome 17
caggaaacagctatgaccgagtttgagaccttggtgaaactag

SEQ ID NO: 11 - DNA, 40 bp, Artificial Sequence
  Sense and antisense primer for the genome section 39.394.075 - 39.569.394 on chromosome 17
tgtaaaacgacggccagtaacaaacgcgtcatgaaggtag

SEQ ID NO: 12 - DNA, 40 bp, Artificial Sequence
  Sense and antisense primer for the genome section 39.394.075 - 39.569.394 on chromosome 17
caggaaacagctatgaccaagagcaggtttgaggtgtgag

SEQ ID NO: 13 - DNA, 40 bp, Artificial Sequence
  Sense and antisense primer for the genome section 39.394.075 - 39.569.394 on chromosome 17
tgtaaaacgacggccagtcagtgcattacattacggggtg

SEQ ID NO: 14 - DNA, 43 bp, Artificial Sequence
  Sense and antisense primer for the genome section 39.394.075 - 39.569.394 on chromosome 17
caggaaacagctatgaccaagtcagtcgctaaaaccactaatt

SEQ ID NO: 15 - DNA, 43 bp, Artificial Sequence
  Sense and antisense primer for the genome section 39.394.075 - 39.569.394 on chromosome 17
tgtaaaacgacggccagttggcctacagagtaatagaatgaag

SEQ ID NO: 16 - DNA, 42 bp, Artificial Sequence
  Sense and antisense primer for the genome section 39.394.075 - 39.569.394 on chromosome 17
caggaaacagctatgaccccccatacatttctatccttgagt

SEQ ID NO: 17 - DNA, 43 bp, Artificial Sequence
  Sense and antisense primer for the genome section 39.394.075 - 39.569.394 on chromosome 17
tgtaaaacgacggccagttgagaattcatgatattgctgcact

SEQ ID NO: 18 - DNA, 41 bp, Artificial Sequence
  Sense and antisense primer for the genome section 39.394.075 - 39.569.394 on chromosome 17
caggaaacagctatgaccgataagcccctgaattgtcgatg

SEQ ID NO: 19 - DNA, 42 bp, Artificial Sequence
  Sense and antisense primer for the genome section 39.394.075 - 39.569.394 on chromosome 17
tgtaaaacgacggccagtttctttgatctgggatgaagacag

SEQ ID NO: 20 - DNA, 41 bp, Artificial Sequence
  Sense and antisense primer for the genome section 39.394.075 - 39.569.394 on chromosome 17
caggaaacagctatgaccaggttggagtcagcatagagttg

SEQ ID NO: 21 - DNA, 43 bp, Artificial Sequence
  Sense and antisense primer for the genome section 39.394.075 - 39.569.394 on chromosome 17
tgtaaaacgacggccagtatatcaaagtaaggccgcagaattt

SEQ ID NO: 22 - DNA, 43 bp, Artificial Sequence
  Sense and antisense primer for the genome section 39.394.075 - 39.569.394 on chromosome 17
caggaaacagctatgaccaaacatgttttcagatgcagtaagg

SEQ ID NO: 23 - DNA, 43 bp, Artificial Sequence
  Sense and antisense primer for the genome section 39.394.075 - 39.569.394 on chromosome 17
tgtaaaacgacggccagtgattacaagtcaatacctccacaga

SEQ ID NO: 24 - DNA, 40 bp, Artificial Sequence
  Sense and antisense primer for the genome section 39.394.075 - 39.569.394 on chromosome 17
caggaaacagctatgaccatgtatatgaagggctgggcag

SEQ ID NO: 25 - DNA, 44 bp, Artificial Sequence
  Sense and antisense primer for the genome section 39.394.075 - 39.569.394 on chromosome 17
tgtaaaacgacggccagtagtattttaactttctttctggcaca

SEQ ID NO: 26 - DNA, 43 bp, Artificial Sequence
  Sense and antisense primer for the genome section 39.394.075 - 39.569.394 on chromosome 17
caggaaacagctatgaccgacaagtaacacaatcttatctgca

SEQ ID NO: 27 - DNA, 43 bp, Artificial Sequence
  Sense and antisense primer for the genome section 39.394.075 - 39.569.394 on chromosome 17
tgtaaaacgacggccagtagctgggactattctgataagattt

SEQ ID NO: 28 - DNA, 43 bp, Artificial Sequence
  Sense and antisense primer for the genome section 39.394.075 - 39.569.394 on chromosome 17
caggaaacagctatgacctgtcatagataaactgaagcatggg

SEQ ID NO: 29 - DNA, 41 bp, Artificial Sequence
  Sense and antisense primer for the genome section 39.394.075 - 39.569.394 on chromosome 17
tgtaaaacgacggccagtgttctgagaggatgacatggaag

SEQ ID NO: 30 - DNA, 43 bp, Artificial Sequence
  Sense and antisense primer for the genome section 39.394.075 - 39.569.394 on chromosome 17
caggaaacagctatgaccaatcagaaggttcatcaagttccaa

SEQ ID NO: 31 - DNA, 43 bp, Artificial Sequence
  Sense and antisense primer for the genome section 39.394.075 - 39.569.394 on chromosome 17
tgtaaaacgacggccagtctggtatactgactgtgagaggaag

SEQ ID NO: 32 - DNA, 42 bp, Artificial Sequence
  Sense and antisense primer for the genome section 39.394.075 - 39.569.394 on chromosome 17
caggaaacagctatgaccaagtttaagggcaataaccaagcc

SEQ ID NO: 33 - DNA, 41 bp, Artificial Sequence
  Sense and antisense primer for the genome section 39.394.075 - 39.569.394 on chromosome 17
tgtaaaacgacggccagtccactttggctgctttcatcaaa

SEQ ID NO: 34 - DNA, 43 bp, Artificial Sequence
  Sense and antisense primer for the genome section 39.394.075 - 39.569.394 on chromosome 17
caggaaacagctatgaccaagaagtcatcaagattaccacctg

SEQ ID NO: 35 - DNA, 42 bp, Artificial Sequence
  Sense and antisense primer for the genome section 39.394.075 - 39.569.394 on chromosome 17
tgtaaaacgacggccagttttctaaagggctgcttccataaa

SEQ ID NO: 36 - DNA, 43 bp, Artificial Sequence
  Sense and antisense primer for the genome section 39.394.075 - 39.569.394 on chromosome 17
caggaaacagctatgaccttgtgtgattcataagaggttgaca

SEQ ID NO: 37 - DNA, 43 bp, Artificial Sequence
  Sense and antisense primer for the genome section 39.394.075 - 39.569.394 on chromosome 17
tgtaaaacgacggccagtacatctctaaatgggactcagatga

SEQ ID NO: 38 - DNA, 42 bp, Artificial Sequence
  Sense and antisense primer for the genome section 39.394.075 - 39.569.394 on chromosome 17
caggaaacagctatgacccttttagattctcctgggcttctc

SEQ ID NO: 39 - DNA, 42 bp, Artificial Sequence
  Sense and antisense primer for the genome section 39.394.075 - 39.569.394 on chromosome 17
tgtaaaacgacggccagtgactgaaattctctctccctcctt

SEQ ID NO: 40 - DNA, 44 bp, Artificial Sequence
  Sense and antisense primer for the genome section 39.394.075 - 39.569.394 on chromosome 17
caggaaacagctatgaccagtagttggagtaataacatagagca

SEQ ID NO: 41 - DNA, 43 bp, Artificial Sequence
  Sense and antisense primer for the genome section 39.394.075 - 39.569.394 on chromosome 17
tgtaaaacgacggccagtatagttcttttgacacagcttccaa

SEQ ID NO: 42 - DNA, 43 bp, Artificial Sequence
  Sense and antisense primer for the genome section 39.394.075 - 39.569.394 on chromosome 17
caggaaacagctatgacctgttagctaggttaggaaatcacta

SEQ ID NO: 43 - DNA, 43 bp, Artificial Sequence
  Sense and antisense primer for the genome section 39.394.075 - 39.569.394 on chromosome 17
tgtaaaacgacggccagtgcagtcttgaagaaacttatctgac

SEQ ID NO: 44 - DNA, 43 bp, Artificial Sequence
  Sense and antisense primer for the genome section 39.394.075 - 39.569.394 on chromosome 17
caggaaacagctatgaccatatcaagtcagaatccaacccttc

SEQ ID NO: 45 - DNA, 43 bp, Artificial Sequence
  Sense and antisense primer for the genome section 39.394.075 - 39.569.394 on chromosome 17
tgtaaaacgacggccagtgtacaaactctcaaaagctcactct

SEQ ID NO: 46 - DNA, 42 bp, Artificial Sequence
  Sense and antisense primer for the genome section 39.394.075 - 39.569.394 on chromosome 17
caggaaacagctatgaccctatccccagtttctcgatctttg

SEQ ID NO: 47 - DNA, 44 bp, Artificial Sequence
  Sense and antisense primer for the genome section 39.394.075 - 39.569.394 on chromosome 17
tgtaaaacgacggccagtcataatggttctgtcttgtaattggg

SEQ ID NO: 48 - DNA, 43 bp, Artificial Sequence
  Sense and antisense primer for the genome section 39.394.075 - 39.569.394 on chromosome 17
caggaaacagctatgaccatgttcttctcatctactctccagc

SEQ ID NO: 49 - DNA, 43 bp, Artificial Sequence
  Sense and antisense primer for the genome section 39.394.075 - 39.569.394 on chromosome 17
tgtaaaacgacggccagttgactggcttcactactattaaagc

SEQ ID NO: 50 - DNA, 43 bp, Artificial Sequence
  Sense and antisense primer for the genome section 39.394.075 - 39.569.394 on chromosome 17
caggaaacagctatgaccttgaggaaaatggttaaagctgatt

SEQ ID NO: 51 - DNA, 40 bp, Artificial Sequence
  Sense and antisense primer for the genome section 39.394.075 - 39.569.394 on chromosome 17
tgtaaaacgacggccagtcaacacttgtgggtatacagca

SEQ ID NO: 52 - DNA, 43 bp, Artificial Sequence
  Sense and antisense primer for the genome section 39.394.075 - 39.569.394 on chromosome 17
caggaaacagctatgacctattttgctgctactcgtttctttt

SEQ ID NO: 53 - DNA, 43 bp, Artificial Sequence
  Sense and antisense primer for the genome section 39.394.075 - 39.569.394 on chromosome 17
tgtaaaacgacggccagtgatagagtagggtgaaaagctatgc

SEQ ID NO: 54 - DNA, 43 bp, Artificial Sequence
  Sense and antisense primer for the genome section 39.394.075 - 39.569.394 on chromosome 17
caggaaacagctatgacctgctttgactttaaatttctcaccc

SEQ ID NO: 55 - DNA, 43 bp, Artificial Sequence
  Sense and antisense primer for the genome section 39.394.075 - 39.569.394 on chromosome 17
tgtaaaacgacggccagtcttgttaaagcctttggatgatgtc

SEQ ID NO: 56 - DNA, 43 bp, Artificial Sequence
  Sense and antisense primer for the genome section 39.394.075 - 39.569.394 on chromosome 17
caggaaacagctatgacccagctaaccctaatcagtgaacttt

SEQ ID NO: 57 - DNA, 43 bp, Artificial Sequence
  Sense and antisense primer for the genome section 39.394.075 - 39.569.394 on chromosome 17
tgtaaaacgacggccagttccccaggtatcttatattaccctt

SEQ ID NO: 58 - DNA, 43 bp, Artificial Sequence
  Sense and antisense primer for the genome section 39.394.075 - 39.569.394 on chromosome 17
caggaaacagctatgacctatatggattttgcatggatggaca

SEQ ID NO: 59 - DNA, 43 bp, Artificial Sequence
  Sense and antisense primer for the genome section 39.394.075 - 39.569.394 on chromosome 17
tgtaaaacgacggccagtctcctaaagttgaaaccaccatgta

SEQ ID NO: 60 - DNA, 43 bp, Artificial Sequence
  Sense and antisense primer for the genome section 39.394.075 - 39.569.394 on chromosome 17
caggaaacagctatgacctaacttgtgtgtcatcttgccatta

SEQ ID NO: 61 - DNA, 42 bp, Artificial Sequence
  Sense and antisense primer for the genome section 39.394.075 - 39.569.394 on chromosome 17
tgtaaaacgacggccagtagaactatgcccaaaatgctaagg

SEQ ID NO: 62 - DNA, 43 bp, Artificial Sequence
  Sense and antisense primer for the genome section 39.394.075 - 39.569.394 on chromosome 17
caggaaacagctatgacctttagattgtcactataccctcctt

SEQ ID NO: 63 - DNA, 41 bp, Artificial Sequence
  Sense and antisense primer for the genome section 39.394.075 - 39.569.394 on chromosome 17
tgtaaaacgacggccagtctcctttcccctacagtcactta

SEQ ID NO: 64 - DNA, 43 bp, Artificial Sequence
  Sense and antisense primer for the genome section 39.394.075 - 39.569.394 on chromosome 17
caggaaacagctatgaccatctggagattttaatttgcgagct

SEQ ID NO: 65 - DNA, 40 bp, Artificial Sequence
  Sense and antisense primer for the genome section 39.394.075 - 39.569.394 on chromosome 17
tgtaaaacgacggccagtgtgaaacggaggcaagaaaggg

SEQ ID NO: 66 - DNA, 40 bp, Artificial Sequence
  Sense and antisense primer for the genome section 39.394.075 - 39.569.394 on chromosome 17
caggaaacagctatgaccgcgagggaagaaatgagagttc

SEQ ID NO: 67 - DNA, 40 bp, Artificial Sequence
  Sense and antisense primer for the genome section 39.394.075 - 39.569.394 on chromosome 17
tgtaaaacgacggccagtcttcttcaggtcaggggaaagg

SEQ ID NO: 68 - DNA, 43 bp, Artificial Sequence
  Sense and antisense primer for the genome section 39.394.075 - 39.569.394 on chromosome 17
caggaaacagctatgaccatagtcatcagtctcctcattcgaa

SEQ ID NO: 69 - DNA, 42 bp, Artificial Sequence
  Sense and antisense primer for the genome section 39.394.075 - 39.569.394 on chromosome 17
tgtaaaacgacggccagtacaaagaccggagtaaaagtcatc

SEQ ID NO: 70 - DNA, 42 bp, Artificial Sequence
  Sense and antisense primer for the genome section 39.394.075 - 39.569.394 on chromosome 17
caggaaacagctatgacccagacaatgttaacacgctgaatg

SEQ ID NO: 71 - DNA, 43 bp, Artificial Sequence
  Sense and antisense primer for the genome section 39.394.075 - 39.569.394 on chromosome 17
tgtaaaacgacggccagtcagaattcgagaagtgaagagtaca

SEQ ID NO: 72 - DNA, 43 bp, Artificial Sequence
  Sense and antisense primer for the genome section 39.394.075 - 39.569.394 on chromosome 17
caggaaacagctatgaccacaaaatttcaaactagcatgcctt

SEQ ID NO: 73 - DNA, 43 bp, Artificial Sequence
  Sense and antisense primer for the genome section 39.394.075 - 39.569.394 on chromosome 17
tgtaaaacgacggccagtaacttaaaatgatctgccccataga

SEQ ID NO: 74 - DNA, 43 bp, Artificial Sequence; identical to SEQ ID NO: 73
  Sense and antisense primer for the genome section 39.394.075 - 39.569.394 on chromosome 17

SEQ ID NO: 75 - DNA, 43 bp, Artificial Sequence; identical to SEQ ID NO: 73
  Sense and antisense primer for the genome section 39.394.075 - 39.569.394 on chromosome 17

SEQ ID NO: 76 - DNA, 43 bp, Artificial Sequence
  Sense and antisense primer for the genome section 39.394.075 - 39.569.394 on chromosome 17
caggaaacagctatgaccccatagtaaaggtgggttatatcca

SEQ ID NO: 77 - DNA, 43 bp, Artificial Sequence
  Sense and antisense primer for the genome section 39.394.075 - 39.569.394 on chromosome 17
tgtaaaacgacggccagtagtccattcatttaaaactggcttt

SEQ ID NO: 78 - DNA, 43 bp, Artificial Sequence
  Sense and antisense primer for the genome section 39.394.075 - 39.569.394 on chromosome 17
caggaaacagctatgacctgttccctgtgctttcaaatcttta

SEQ ID NO: 79 - DNA, 42 bp, Artificial Sequence
  Sense and antisense primer for the genome section 39.394.075 - 39.569.394 on chromosome 17
tgtaaaacgacggccagtccaaaggagacagaaacatcagaa

SEQ ID NO: 80 - DNA, 43 bp, Artificial Sequence
  Sense and antisense primer for the genome section 39.394.075 - 39.569.394 on chromosome 17
caggaaacagctatgaccgttaaccaggcctcaaattctaatt

SEQ ID NO: 81 - DNA, 43 bp, Artificial Sequence
  Sense and antisense primer for the genome section 39.394.075 - 39.569.394 on chromosome 17
tgtaaaacgacggccagtccacagattcagttcagattccaaa

SEQ ID NO: 82 - DNA, 43 bp, Artificial Sequence
  Sense and antisense primer for the genome section 39.394.075 - 39.569.394 on chromosome 17
caggaaacagctatgaccggttatgtcctaccagttcaggata

SEQ ID NO: 83 - DNA, 43 bp, Artificial Sequence
  Sense and antisense primer for the genome section 39.394.075 - 39.569.394 on chromosome 17
tgtaaaacgacggccagtgcatgttcatctctcccagtatttc

SEQ ID NO: 84 - DNA, 43 bp, Artificial Sequence
  Sense and antisense primer for the genome section 39.394.075 - 39.569.394 on chromosome 17
caggaaacagctatgacctagtacaacagactaactcatgtct

SEQ ID NO: 85 - DNA, 43 bp, Artificial Sequence
  Sense and antisense primer for the genome section 39.394.075 - 39.569.394 on chromosome 17
tgtaaaacgacggccagttctacaaaaccccactgactactat

SEQ ID NO: 86 - DNA, 43 bp, Artificial Sequence
  Sense and antisense primer for the genome section 39.394.075 - 39.569.394 on chromosome 17
caggaaacagctatgaccaattcacaaagtatcactgcaactc

SEQ ID NO: 87 - DNA, 43 bp, Artificial Sequence
  Sense and antisense primer for the genome section 39.394.075 - 39.569.394 on chromosome 17
tgtaaaacgacggccagttaaccttttcgtaaccaggcattat

SEQ ID NO: 88 - DNA, 43 bp, Artificial Sequence
  Sense and antisense primer for the genome section 39.394.075 - 39.569.394 on chromosome 17
tgtaaaacgacggccagtgacttaatgggactgctagaatctg

SEQ ID NO: 89 - DNA, 44 bp, Artificial Sequence
  Sense and antisense primer for the genome section 39.394.075 - 39.569.394 on chromosome 17
caggaaacagctatgacctctgatattaccaatgcagtataggc

SEQ ID NO: 90 - DNA, 43 bp, Artificial Sequence
  Sense and antisense primer for the genome section 39.394.075 - 39.569.394 on chromosome 17
caggaaacagctatgaccacacgattgacacatcctgaattta

SEQ ID NO: 91 - DNA, 43 bp, Artificial Sequence
  Sense and antisense primer for the genome section 39.394.075 - 39.569.394 on chromosome 17
tgtaaaacgacggccagtgctttctctgactcataattcccag

SEQ ID NO: 92 - DNA, 43 bp, Artificial Sequence
  Sense and antisense primer for the genome section 39.394.075 - 39.569.394 on chromosome 17
caggaaacagctatgacccggcataaactgataatttggtgac

SEQ ID NO: 93 - DNA, 44 bp, Artificial Sequence
  Sense and antisense primer for the genome section 39.394.075 - 39.569.394 on chromosome 17
tgtaaaacgacggccagtgccctcttaagtctttctcaatctag

SEQ ID NO: 94 - DNA, 41 bp, Artificial Sequence
  Sense and antisense primer for the genome section 39.394.075 - 39.569.394 on chromosome 17
caggaaacagctatgacctcttcagagttatagagccgagc

SEQ ID NO: 95 - DNA, 43 bp, Artificial Sequence
  Sense and antisense primer for the genome section 39.394.075 - 39.569.394 on chromosome 17
tgtaaaacgacggccagttcttcatctcctacttctgaacagt

SEQ ID NO: 96 - DNA, 42 bp, Artificial Sequence
  Sense and antisense primer for the genome section 39.394.075 - 39.569.394 on chromosome 17
caggaaacagctatgaccggaaatctggccaaaagtttatcg

SEQ ID NO: 97 - DNA, 43 bp, Artificial Sequence
  Sense and antisense primer for the genome section 39.394.075 - 39.569.394 on chromosome 17
tgtaaaacgacggccagtaaaagactcttgactccctttctct

SEQ ID NO: 98 - DNA, 42 bp, Artificial Sequence
  Sense and antisense primer for the genome section 39.394.075 - 39.569.394 on chromosome 17
caggaaacagctatgacccatctgagctatgaatggaagagc

SEQ ID NO: 99 - DNA, 43 bp, Artificial Sequence
  Sense and antisense primer for the genome section 39.394.075 - 39.569.394 on chromosome 17
tgtaaaacgacggccagttcttctcatctcacagtttacccta

SEQ ID NO: 100 - DNA, 43 bp, Artificial Sequence
  Sense and antisense primer for the genome section 39.394.075 - 39.569.394 on chromosome 17
caggaaacagctatgaccctacactgtgttgccatgataaact

SEQ ID NO: 101 - DNA, 43 bp, Artificial Sequence
  Sense and antisense primer for the genome section 39.394.075 - 39.569.394 on chromosome 17
tgtaaaacgacggccagtgatgagcaagttgatatccagtcat

SEQ ID NO: 102 - DNA, 43 bp, Artificial Sequence
  Sense and antisense primer for the genome section 39.394.075 - 39.569.394 on chromosome 17
caggaaacagctatgaccaaaggggaaatatgtaagggaatgc

SEQ ID NO: 103 - DNA, 43 bp, Artificial Sequence
  Sense and antisense primer for the genome section 39.394.075 - 39.569.394 on chromosome 17
tgtaaaacgacggccagtctccatccaaaacttctcgaaaaga

SEQ ID NO: 104 - DNA, 43 bp, Artificial Sequence
  Sense and antisense primer for the genome section 39.394.075 - 39.569.394 on chromosome 17
caggaaacagctatgaccttttgtcccgaagaattggtcataa

SEQ ID NO: 105 - DNA, 43 bp, Artificial Sequence
  Sense and antisense primer for the genome section 39.394.075 - 39.569.394 on chromosome 17
tgtaaaacgacggccagtgaagtcacttttatgcacaatccag

SEQ ID NO: 106 - DNA, 43 bp, Artificial Sequence
  Sense and antisense primer for the genome section 39.394.075 - 39.569.394 on chromosome 17
caggaaacagctatgaccagaactgccaatatattctgcatgt

SEQ ID NO: 107 - DNA, 40 bp, Artificial Sequence
  Sense and antisense primer for the genome section 39.394.075 - 39.569.394 on chromosome 17
tgtaaaacgacggccagtctgatgaaaacccaagagccag

SEQ ID NO: 108 - DNA, 43 bp, Artificial Sequence
  Sense and antisense primer for the genome section 39.394.075 - 39.569.394 on chromosome 17
caggaaacagctatgaccgtctgataatggtttgtttggaagt

SEQ ID NO: 109 - DNA, 43 bp, Artificial Sequence
  Sense and antisense primer for the genome section 39.394.075 - 39.569.394 on chromosome 17
tgtaaaacgacggccagtcctggaagatacaatcaagaaaacc

SEQ ID NO: 110 - DNA, 43 bp, Artificial Sequence
  Sense and antisense primer for the genome section 39.394.075 - 39.569.394 on chromosome 17
caggaaacagctatgacccatatactttctgcaccacctcata

SEQ ID NO: 111 - DNA, 43 bp, Artificial Sequence
  Sense and antisense primer for the genome section 39.394.075 - 39.569.394 on chromosome 17
tgtaaaacgacggccagtgacctcaagtgttagttctgttgaa

SEQ ID NO: 112 - DNA, 43 bp, Artificial Sequence
  Sense and antisense primer for the genome section 39.394.075 - 39.569.394 on chromosome 17
caggaaacagctatgacctgtatagtacccacccaagttattt

SEQ ID NO: 113 - DNA, 42 bp, Artificial Sequence
  Sense and antisense primer for the genome section 39.394.075 - 39.569.394 on chromosome 17
tgtaaaacgacggccagtcagcaggatgactctcaattttct

SEQ ID NO: 114 - DNA, 42 bp, Artificial Sequence
  Sense and antisense primer for the genome section 39.394.075 - 39.569.394 on chromosome 17
caggaaacagctatgaccttctccaaactttagtcacgtctc

SEQ ID NO: 115 - DNA, 43 bp, Artificial Sequence
  Sense and antisense primer for the genome section 39.394.075 - 39.569.394 on chromosome 17
tgtaaaacgacggccagtgtaaaatgtgttagccaagtccatg

SEQ ID NO: 116 - DNA, 43 bp, Artificial Sequence
  Sense and antisense primer for the genome section 39.394.075 - 39.569.394 on chromosome 17
caggaaacagctatgaccgcacatgtgattataaacacaaaca

SEQ ID NO: 117 - DNA, 41 bp, Artificial Sequence
  Sense and antisense primer for the genome section 39.394.075 - 39.569.394 on chromosome 17
tgtaaaacgacggccagtgcaacttttatcccagcctgaag

SEQ ID NO: 118 - DNA, 43 bp, Artificial Sequence
  Sense and antisense primer for the genome section 39.394.075 - 39.569.394 on chromosome 17
caggaaacagctatgaccctgaagtctctgggttagtaaggaa

SEQ ID NO: 119 - DNA, 43 bp, Artificial Sequence
  Sense and antisense primer for the genome section 39.394.075 - 39.569.394 on chromosome 17
tgtaaaacgacggccagtgtattccttgctcacttgctactag

SEQ ID NO: 120 - DNA, 43 bp, Artificial Sequence
  Sense and antisense primer for the genome section 39.394.075 - 39.569.394 on chromosome 17
caggaaacagctatgaccagtggagtaaactttaaggatgagt

SEQ ID NO: 121 - DNA, 43 bp, Artificial Sequence
  Sense and antisense primer for the genome section 39.394.075 - 39.569.394 on chromosome 17
tgtaaaacgacggccagtctctttgaggcatttgtttggaaaa

SEQ ID NO: 122 - DNA, 43 bp, Artificial Sequence
  Sense and antisense primer for the genome section 39.394.075 - 39.569.394 on chromosome 17
caggaaacagctatgaccactcacatagaaatagaagccagaa

SEQ ID NO: 123 - DNA, 43 bp, Artificial Sequence
  Sense and antisense primer for the genome section 39.394.075 - 39.569.394 on chromosome 17
tgtaaaacgacggccagtgactgttttcccctgttaatgagat

SEQ ID NO: 124 - DNA, 43 bp, Artificial Sequence
  Sense and antisense primer for the genome section 39.394.075 - 39.569.394 on chromosome 17
caggaaacagctatgaccattgatgactgctgataaccaaaat

SEQ ID NO: 125 - DNA, 43 bp, Artificial Sequence
  Sense and antisense primer for the genome section 39.394.075 - 39.569.394 on chromosome 17
tgtaaaacgacggccagtatatacttaagcctccattccctca

SEQ ID NO: 126 - DNA, 43 bp, Artificial Sequence
  Sense and antisense primer for the genome section 39.394.075 - 39.569.394 on chromosome 17
caggaaacagctatgaccttatcgttcctatggacagttgaag

SEQ ID NO: 127 - DNA, 43 bp, Artificial Sequence
  Sense and antisense primer for the genome section 39.394.075 - 39.569.394 on chromosome 17
tgtaaaacgacggccagttgatttaggttcctgacactgattc

SEQ ID NO: 128 - DNA, 43 bp, Artificial Sequence
  Sense and antisense primer for the genome section 39.394.075 - 39.569.394 on chromosome 17
caggaaacagctatgaccaatctcagcaaaggtaggtacacta

SEQ ID NO: 129 - DNA, 43 bp, Artificial Sequence
  Sense and antisense primer for the genome section 39.394.075 - 39.569.394 on chromosome 17
tgtaaaacgacggccagtttgtttccaaaatctttcccatctc

SEQ ID NO: 130 - DNA, 43 bp, Artificial Sequence
  Sense and antisense primer for the genome section 39.394.075 - 39.569.394 on chromosome 17
caggaaacagctatgaccaatctaggttagtaaggtcctcagc

SEQ ID NO: 131 - DNA, 43 bp, Artificial Sequence
  Sense and antisense primer for the genome section 39.394.075 - 39.569.394 on chromosome 17
tgtaaaacgacggccagtttcagtacctcaatttcttaggcac

SEQ ID NO: 132 - DNA, 43 bp, Artificial Sequence
  Sense and antisense primer for the genome section 39.394.075 - 39.569.394 on chromosome 17
caggaaacagctatgaccgtaggttgaggatccagatagacat

SEQ ID NO: 133 - DNA, 45 bp, Artificial Sequence
  Sense and antisense primer for the genome section 39.394.075 - 39.569.394 on chromosome 17
tgtaaaacgacggccagtgcttttcatttgtgttatgtagggtac

SEQ ID NO: 134 - DNA, 43 bp, Artificial Sequence
  Sense and antisense primer for the genome section 39.394.075 - 39.569.394 on chromosome 17
caggaaacagctatgaccaaagtccatccatttacaggtccta

SEQ ID NO: 135 - DNA, 43 bp, Artificial Sequence
  Sense and antisense primer for the genome section 39.394.075 - 39.569.394 on chromosome 17
tgtaaaacgacggccagttaagaagtccaccatatagcagtca

SEQ ID NO: 136 - DNA, 43 bp, Artificial Sequence
  Sense and antisense primer for the genome section 39.394.075 - 39.569.394 on chromosome 17
caggaaacagctatgaccgttctcccttcttacccttatttcc

SEQ ID NO: 137 - DNA, 43 bp, Artificial Sequence
  Sense and antisense primer for the genome section 39.394.075 - 39.569.394 on chromosome 17
tgtaaaacgacggccagtagattcacagttcatttcaggaaga

SEQ ID NO: 138 - DNA, 43 bp, Artificial Sequence
  Sense and antisense primer for the genome section 39.394.075 - 39.569.394 on chromosome 17
caggaaacagctatgaccaaaatgagtggcagaggaatactac

SEQ ID NO: 139 - DNA, 43 bp, Artificial Sequence
  Sense and antisense primer for the genome section 39.394.075 - 39.569.394 on chromosome 17
tgtaaaacgacggccagttaggactgatagggaccaagattac

SEQ ID NO: 140 - DNA, 43 bp, Artificial Sequence
  Sense and antisense primer for the genome section 39.394.075 - 39.569.394 on chromosome 17
caggaaacagctatgaccatatgttgacttacagattccccac

SEQ ID NO: 141 - DNA, 43 bp, Artificial Sequence
  Sense and antisense primer for the genome section 39.394.075 - 39.569.394 on chromosome 17
tgtaaaacgacggccagtaaaccaaaggaaagaaataatcgga

SEQ ID NO: 142 - DNA, 42 bp, Artificial Sequence
  Sense and antisense primer for the genome section 39.394.075 - 39.569.394 on chromosome 17
caggaaacagctatgaccccctcttatcaagctttcatcacc

SEQ ID NO: 143 - DNA, 44 bp, Artificial Sequence
  Sense and antisense primer for the genome section 39.394.075 - 39.569.394 on chromosome 17
tgtaaaacgacggccagttgataggatgtgtcaatgttcagtat

SEQ ID NO: 144 - DNA, 41 bp, Artificial Sequence
  Sense and antisense primer for the genome section 39.394.075 - 39.569.394 on chromosome 17
caggaaacagctatgaccttttgaaacagagaggacttggc

SEQ ID NO: 145 - DNA, 43 bp, Artificial Sequence
  Sense and antisense primer for the genome section 39.394.075 - 39.569.394 on chromosome 17
tgtaaaacgacggccagtggacaaaacaatttataggaagcca

SEQ ID NO: 146 - DNA, 43 bp, Artificial Sequence
  Sense and antisense primer for the genome section 39.394.075 - 39.569.394 on chromosome 17
caggaaacagctatgaccttaagaggaaaagaagttgaggcag

SEQ ID NO: 147 - DNA, 43 bp, Artificial Sequence
  Sense and antisense primer for the genome section 39.394.075 - 39.569.394 on chromosome 17
tgtaaaacgacggccagtggaattcaccctaaatctcaactga

SEQ ID NO: 148 - DNA, 43 bp, Artificial Sequence
  Sense and antisense primer for the genome section 39.394.075 - 39.569.394 on chromosome 17
caggaaacagctatgaccactctttctaatctgcccaatgatg

SEQ ID NO: 149 - DNA, 43 bp, Artificial Sequence
  Sense and antisense primer for the genome section 39.394.075 - 39.569.394 on chromosome 17
tgtaaaacgacggccagtagatcccagtacatatcctttacgt

SEQ ID NO: 150 - DNA, 43 bp, Artificial Sequence
  Sense and antisense primer for the genome section 39.394.075 - 39.569.394 on chromosome 17
caggaaacagctatgacctgtaaaggaaaatggcaggttacat

SEQ ID NO: 151 - DNA, 43 bp, Artificial Sequence
  Sense and antisense primer for the genome section 39.394.075 - 39.569.394 on chromosome 17
tgtaaaacgacggccagttctcactacctctaatcagcttctt

SEQ ID NO: 152 - DNA, 41 bp, Artificial Sequence
  Sense and antisense primer for the genome section 39.394.075 - 39.569.394 on chromosome 17
caggaaacagctatgacctcctagtactctcccaggatagc

SEQ ID NO: 153 - DNA, 43 bp, Artificial Sequence
  Sense and antisense primer for the genome section 39.394.075 - 39.569.394 on chromosome 17
tgtaaaacgacggccagtcttttgagcgaatggttttatgtgt

SEQ ID NO: 154 - DNA, 43 bp, Artificial Sequence
  Sense and antisense primer for the genome section 39.394.075 - 39.569.394 on chromosome 17
caggaaacagctatgacctgcatacttactgcttgactacatc

SEQ ID NO: 155 - DNA, 42 bp, Artificial Sequence
  Sense and antisense primer for the genome section 39.394.075 - 39.569.394 on chromosome 17
tgtaaaacgacggccagtccaattaaaactcatgccccttag

SEQ ID NO: 156 - DNA, 43 bp, Artificial Sequence
  Sense and antisens primer for the genome section 39.394.075 - 39.569.394 on chromosome 17
caggaaacagctatgacccttgaattctataggtttgtgctgg

SEQ ID NO: 157 - DNA, 41 bp, Artificial Sequence
  Sense and antisense primer for the genome section 39.394.075 - 39.569.394 on chromosome 17
tgtaaaacgacggccagtctaagggacatgacagaggttct

SEQ ID NO: 158 - DNA, 42 bp, Artificial Sequence
  Sense and antisense primer for the genome section 39.394.075 - 39.569.394 on chromosome 17
caggaaacagctatgacccttacggtgtccctagcattttag

SEQ ID NO: 159 - DNA, 42 bp, Artificial Sequence
  Sense and antisense primer for the genome section 39.394.075 - 39.569.394 on chromosome 17
tgtaaaacgacggccagtgtcctttacctatgaagtgctagg

SEQ ID NO: 160 - DNA, 41 bp, Artificial Sequence
  Sense and antisense primer for the genome section 39.394.075 - 39.569.394 on chromosome 17
caggaaacagctatgacccaccatgccctagtgtctaattt

SEQ ID NO: 161 - DNA, 42 bp, Artificial Sequence
  Sense and antisense primer for the genome section 39.394.075 - 39.569.394 on chromosome 17
tgtaaaacgacggccagtacgttttcacctgccatttaattg

SEQ ID NO: 162 - DNA, 42 bp, Artificial Sequence
  Sense and antisense primer for the genome section 39.394.075 - 39.569.394 on chromosome 17
caggaaacagctatgacctgtcttctctctacaaggacctca

SEQ ID NO: 163 - DNA, 43 bp, Artificial Sequence
  Sense and antisense primer for the genome section 39.394.075 - 39.569.394 on chromosome 17
tgtaaaacgacggccagtgaaaaggaaaggtaggtaggtgttg

SEQ ID NO: 164 - DNA, 43 bp, Artificial Sequence
  Sense and antisense primer for the genome section 39.394.075 - 39.569.394 on chromosome 17
caggaaacagctatgaccggacaagacgtataagactactcct

SEQ ID NO: 165 - DNA, 41 bp, Artificial Sequence
  Sense and antisense primer for the genome section 39.394.075 - 39.569.394 on chromosome 17
tgtaaaacgacggccagtgttgatgtacactggagaaaggg

SEQ ID NO: 166 - DNA, 41 bp, Artificial Sequence
  Sense and antisense primer for the genome section 39.394.075 - 39.569.394 on chromosome 17
caggaaacagctatgaccgttaagaaggcaggcaatgagac

SEQ ID NO: 167 - DNA, 40 bp, Artificial Sequence
  Sense and antisense primer for the genome section 39.394.075 - 39.569.394 on chromosome 17
tgtaaaacgacggccagtgaatttatctaggaccccgtgc

SEQ ID NO: 168 - DNA, 43 bp, Artificial Sequence
  Sense and antisense primer for the genome section 39.394.075 - 39.569.394 on chromosome 17
caggaaacagctatgaccatctgacaggagttacaagaaagga

SEQ ID NO: 169 - DNA, 43 bp, Artificial Sequence
  Sense and antisense primer for the genome section 39.394.075 - 39.569.394 on chromosome 17
tgtaaaacgacggccagtctcagcctctcatttctaaggttta

SEQ ID NO: 170 - DNA, 43 bp, Artificial Sequence
  Sense and antisense primer for the genome section 39.394.075 - 39.569.394 on chromosome 17
caggaaacagctatgaccttcctctctagttaccttcttctcc

SEQ ID NO: 171 - DNA, 43 bp, Artificial Sequence
  Sense and antisense primer for the genome section 39.394.075 - 39.569.394 on chromosome 17
tgtaaaacgacggccagttgttgtggattcctagaaatgaagg

SEQ ID NO: 172 - DNA, 40 bp, Artificial Sequence
  Sense and antisense primer for the genome section 39.394.075 - 39.569.394 on chromosome 17
caggaaacagctatgaccctaccctagcacagatggatgc

SEQ ID NO: 173 - DNA, 41 bp, Artificial Sequence
  Sense and antisense primer for the genome section 39.394.075 - 39.569.394 on chromosome 17
tgtaaaacgacggccagtctaggcaaacacagttactcctc

SEQ ID NO: 174 - DNA, 43 bp, Artificial Sequence
  Sense and antisense primer for the genome section 39.394.075 - 39.569.394 on chromosome 17
caggaaacagctatgaccactctttaccagatccagtataggc

SEQ ID NO: 175 - DNA, 42 bp, Artificial Sequence
  Sense and antisense primer for the genome section 39.394.075 - 39.569.394 on chromosome 17
tgtaaaacgacggccagtgagcctgagttctcatttgcctat

SEQ ID NO: 176 - DNA, 43 bp, Artificial Sequence
  Sense and antisense primer for the genome section 39.394.075 - 39.569.394 on chromosome 17
caggaaacagctatgacccatgattcttttatctctcctgggg

SEQ ID NO: 177 - DNA, 43 bp, Artificial Sequence
  Sense and antisense primer for the genome section 39.394.075 - 39.569.394 on chromosome 17
tgtaaaacgacggccagtctatccctgtttcaactctttccag

SEQ ID NO: 178 - DNA, 41 bp, Artificial Sequence
  Sense and antisense primer for the genome section 39.394.075 - 39.569.394 on chromosome 17
caggaaacagctatgaccccacacattattctaccctgctg

SEQ ID NO: 179 - DNA, 42 bp, Artificial Sequence
  Sense and antisense primer for the genome section 39.394.075 - 39.569.394 on chromosome 17
tgtaaaacgacggccagtcattctatgtgtccagtcttaggc

SEQ ID NO: 180 - DNA, 43 bp, Artificial Sequence
  Sense and antisense primer for the genome section 39.394.075 - 39.569.394 on chromosome 17
caggaaacagctatgaccggtgtcaaactagaagatgtgtagg

SEQ ID NO: 181 - DNA, 43 bp, Artificial Sequence
  Sense and antisense primer for the genome section 39.394.075 - 39.569.394 on chromosome 17
tgtaaaacgacggccagtgagacctatgataagtgagctctct

SEQ ID NO: 182 - DNA, 42 bp, Artificial Sequence
  Sense and antisense primer for the genome section 39.394.075 - 39.569.394 on chromosome 17
caggaaacagctatgacctagcctcagtgtttatatcccaca

SEQ ID NO: 183 - DNA, 43 bp, Artificial Sequence
  Sense and antisense primer for the genome section 39.394.075 - 39.569.394 on chromosome 17
tgtaaaacgacggccagtctagagatacccattctgaacctct

SEQ ID NO: 184 - DNA, 41 bp, Artificial Sequence
  Sense and antisense primer for the genome section 39.394.075 - 39.569.394 on chromosome 17
caggaaacagctatgaccgaaaatgcgttgaagttgagagt

SEQ ID NO: 185 - DNA, 42 bp, Artificial Sequence
  Sense and antisense primer for the genome section 39.394.075 - 39.569.394 on chromosome 17
tgtaaaacgacggccagtgcctaattcttctaacactctgct

SEQ ID NO: 186 - DNA, 42 bp, Artificial Sequence
  Sense and antisense primer for the genome section 39.394.075 - 39.569.394 on chromosome 17
caggaaacagctatgacctgggacacacactatcatagactc

SEQ ID NO: 187 - DNA, 43 bp, Artificial Sequence
  Sense and antisense primer for the genome section 39.394.075 - 39.569.394 on chromosome 17
tgtaaaacgacggccagtccacctctatattcagtcaaaggag

SEQ ID NO: 188 - DNA, 40 bp, Artificial Sequence
  Sense and antisense primer for the genome section 39.394.075 - 39.569.394 on chromosome 17
caggaaacagctatgaccagggacatagactggaagtggc

SEQ ID NO: 189 - DNA, 43 bp, Artificial Sequence
  Sense and antisense primer for the genome section 39.394.075 - 39.569.394 on chromosome 17
tgtaaaacgacggccagttaagttctggaaaaggtctgattgc

SEQ ID NO: 190 - DNA, 41 bp, Artificial Sequence
  Sense and antisense primer for the genome section 39.394.075 - 39.569.394 on chromosome 17
caggaaacagctatgaccgggtgaatctgagtgtcttaggt

SEQ ID NO: 191 - DNA, 43 bp, Artificial Sequence
  Sense and antisense primer for the genome section 39.394.075 - 39.569.394 on chromosome 17
tgtaaaacgacggccagtaggaagtaggttagggaatggtaag

SEQ ID NO: 192 - DNA, 43 bp, Artificial Sequence
  Sense and antisense primer for the genome section 39.394.075 - 39.569.394 on chromosome 17
caggaaacagctatgacccagtgacatcagagaatcaaaggag

SEQ ID NO: 193 - DNA, 41 bp, Artificial Sequence
  Sense and antisense primer for the genome section 39.394.075 - 39.569.394 on chromosome 17
tgtaaaacgacggccagtagaacatctctctggcctaaact

SEQ ID NO: 194 - DNA, 43 bp, Artificial Sequence
  Sense and antisense primer for the genome section 39.394.075 - 39.569.394 on chromosome 17
caggaaacagctatgaccgtttaaccatctctttccctgaagt

SEQ ID NO: 195 - DNA, 43 bp, Artificial Sequence
  Sense and antisense primer for the genome section 39.394.075 - 39.569.394 on chromosome 17
tgtaaaacgacggccagtttaccatctgacctcaaacaatgtt

SEQ ID NO: 196 - DNA, 41 bp, Artificial Sequence
  Sense and antisense primer for the genome section 39.394.075 - 39.569.394 on chromosome 17
caggaaacagctatgacccaatctatgcctgcagtggaatc

SEQ ID NO: 197 - DNA, 43 bp, Artificial Sequence
  Sense and antisense primer for the genome section 39.394.075 - 39.569.394 on chromosome 17
tgtaaaacgacggccagtgccttcttttattcatgcgttgttt

SEQ ID NO: 198 - DNA, 42 bp, Artificial Sequence
  Sense and antisense primer for the genome section 39.394.075 - 39.569.394 on chromosome 17
caggaaacagctatgaccccttctcttcttgtttcctggtaa

(19) United States
(12) Patent Application Publication
(10) Pub. No.: US 2023/0183780 A1
(43) Pub. Date: Jun. 15, 2023

(54) DNA PROBES FOR IN SITU HYBRIDIZATION ON CHROMOSOMES

(71) Applicant: InVivo BioTech Services GmbH, Hennigsdorf - Brandenburg (DE)

(72) Inventors: Wolfgang WEGLÖHNER, Hennigsdorf - Brandenburg (DE); Sabrina SCHINDLER, Hennigsdorf - Brandenburg (DE)

(21) Appl. No.: 16/320,325

(22) PCT Filed: Jul. 25, 2016
(86) PCT No.: PCT/EP2017/067801

§ 371 Date: Jan. 24, 2019

(30) Foreign Application Priority Data
Jul. 25, 2016 (DE) 102016113681.6
Dec. 19, 2016 (DE) 102016124844.4

(51) Int. Cl. C12Q 1/6827; C12Q 1/6841; C12Q 1/6853

(52) U.S. Cl. CPC C12Q 1/6827; C12Q 1/6841; C12Q 1/6853; C12Q 2600/156; C12Q 2600/16

(57) ABSTRACT

Kit, probe mixture and probes for the detection of a chromosome aberration. Genetic probe, obtained by a method comprising the following steps: (a) Examine genomic segments for sequence regions with non-repetitive nucleic acid sequences and select one or more nucleic acid sequences; (b) Design and synthesize primer pairs for a polymerase chain reaction on the non-repetitive nucleic acid sequences, where the primers each have an oligonucleotide sequence which is complementary to the strand or complementary strand of the non-repetitive nucleic acid sequence, and a non-complementary universal linker sequence; (c) Carry out a first PCR and obtain a first mixture (pool A) of nucleic acid fragments; (d) Carry out a multiplex PCR on the mixture (pool A) with the use of primers which hybridize onto the linkers, and obtain a mixture (pool B) with amplified, non-repetitive nucleic acid fragments which are suitable for chromogenic or fluorescence in-situ hybridization of chromosomes (FISH/CISH/ISH).

A sequence listing related to the invention has been submitted concurrently herewith and its contents are incorporated by reference herein.

### FIELD OF THE INVENTION

The invention relates to methods for producing probes for an in-situ hybridization of chromosomes for the diagnosis of chromosome aberrations and DNA probes and probe mixtures thus produced.

### BACKGROUND OF THE INVENTION

Chromosome aberrations can often be observed in tumor cells. They can be determined by G-banding or in-situ hybridization (ISH) with probes for specific genome loci. In multicolor fluorescence in-situ hybridization, several regions of the genome are labeled in different colors; these regions can also be located at a distance from the loci of diseases and breakpoints, so that even complex chromosome aberrations can easily be diagnosed. Some aberrations occur only with specific tumors, for example the Philadelphia chromosome with certain leukemias; others indicate the type of medical treatment, particularly with tumors of the breast. Clinically relevant here are the oncogene ERBB2, which codes for a cell surface receptor, and the CEN17 region for the centromere region of chromosome 17. ISH then provides a means of assessing the aggressiveness of the tumor and a more targeted treatment for the patients. Subgroups of non-Hodgkin's lymphomas can also be distinguished via genetic variations.

Conventional methods of producing ISH probes use BAC clones (Bacterial Artificial Chromosome), YAC clones, cosmids and fosmids. These contain large regions of genomic DNA (up to 500 kilobases) and thus repetitive sequences, pseudogenes and paralogous sequences in addition to the sequences sought. They cause nonspecific hybridizations and background in chromogenic or fluorescence ISH, which makes an evaluation more difficult and sometimes impossible. In CISH (Chromogenic In-Situ Hybridization) in particular, a high background is often unavoidable. CISH probes are usually smaller than FISH (Fluorescence In-Situ Hybridization) probes.

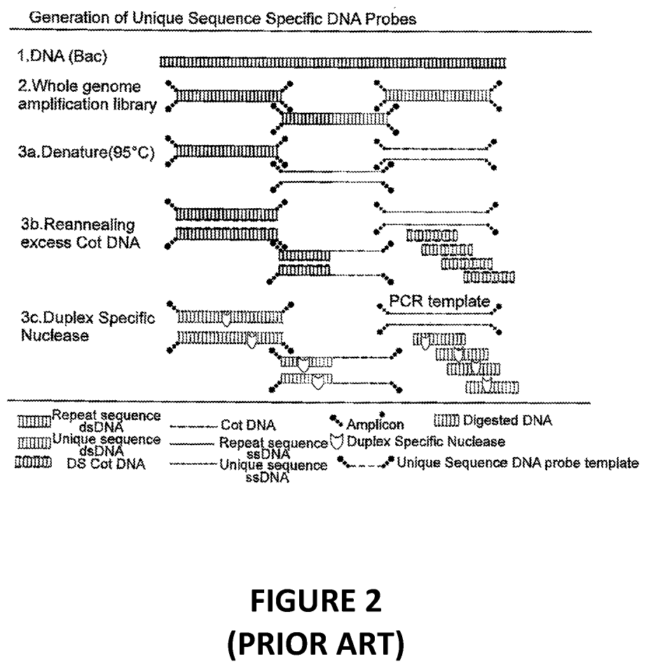

To avoid background caused by repetitive sequences, a high excess of so-called blocking DNA (e.g. Cot-1 DNA, salmon sperm DNA, tRNA, o.a.) is frequently added in the case of BAC probes. Signals from the repetitive sequences still cannot be prevented altogether, however. Hence attempts are also often made “to free” the BAC clones of repetitive sequences; see Swennenhuis J F et al, Construction of repeat-free fluorescence in situ hybridization probes, 2011, Nucleic Acids Research, 2012, Vol. 40, No. 3, e20, doi:10.1093/nar/gkr1123, WO 2007/053245, and the Prior Art in FIG. 2. The BAC clones are randomly fragmented, and the fragments are equipped with linkers and amplified in a PCR. After the addition of an excess of Cot-1DNA, the DNA is dissolved and digested at 65° C. with a double-strand specific nuclease (DSN—duplex-specific nuclease). The amplification and digest steps in the presence of Cot-1DNA are repeated several times, until a probe or a probe mixture “free” from repetitive human sequences is obtained. The removal of the repetitive sequences can also be achieved by other methods; see Craid J M et al, Removal of repetitive sequences from FISH probes using PCR-assisted affinity chromatography, HUMAN GENETICS (Springer, Berlin, DE) Vol. 100, pp. 472-476; US 2004029298, WO 2004/083386; WO 01/06014. But these methods not only involve considerable effort, they are unfortunately also never complete or certain. Neither is it possible to simply secure the probe DNA in plasmids.

ISH probes can also be produced in a PCR (polymerase chain reaction). The PCR takes place on the genome, and specifically on regions which are free from repetitive sequences, and often code for only part of a gene or a domain. Repetitive and Alu sequences can be found everywhere in the human genome and on all chromosomes. U.S. Pat. No. 8,407,013 B2 discloses a computer-assisted sequence analysis and an ab-initio generation of genomic probes by PCR; see Rogan P K et al Sequence-based design of single-copy genomic DNA probes for fluorescence in situ hybridization, Genome Res. (2001) 11(6): 1086-1094. For the Prior Art see also U.S. Pat. Nos. 6,150,160-A, 6,828,097 B1, 7,014,997 B2; US 2000 3002204 A1; EP 1127163; US 2000 30108943 A1; DE 69032920; US 2000 30194718 A1; US 2000 40161773 A1; WO 2004/04097050; WO 2004/083386; EP 1285093; WO 2014/036525-A1, WO 2000/188089; EP 2069537; EP 1127163; EP 1669902; and methods in accordance with DE 10 2010 029 855 A1. The production of probes which are free from repetitive sequences remains problematic, however, since the coverage of these types of probes is often not large. Several megabases are required for clinically relevant ISH probes, however.

The probe must, furthermore, additionally be labeled with radioactive, chromogenic or fluorescent groups. The labeling can be done enzymatically by means of a nick translation reaction, by random priming, or by direct PCR labeling with labeled nucleotides and/or by chemical coupling. The labeling in the amplification reaction is complex, however, because the polymerase chain reaction has to be re-established for every labeling, every fluorescent dye, and every chromogenic group. It also depends on sequence length, chemical structure of the modification, length of the linker between labeling and nucleotide, and also on the polymerase and the condition of the starting material. The Prior Art thus represents a problem.

### SUMMARY OF THE INVENTION

The problem is solved by the method according to claim 1 and by probes in accordance with claims 13 and 14. Advantageous embodiments of the method and the probes can be found in the subclaims and are described in the Examples.

The method comprises the production of directly or indirectly labeled nucleic acids, comprising an analysis of sequences in larger genomic regions for segments with specific sequences and the selection of specific nucleic acid sequences for specific loci; design and synthesis of sense and antisense primer pairs for a polymerase chain reaction on selected, specific nucleic acid sequences, where the synthesized primers each contain a sequence which is complementary to the strand or complementary strand of the non-specific nucleic sequence, and a non-complementary uniform linker sequence, which does not hybridize with the genome under stringent conditions and can facultatively contain a cleavage sequence for a restriction endonuclease; a number of first polymerase chain reactions with the number of sense and antisense primer pairs and, after combining the reaction products, obtaining a first mixture (pool A) of synthesized PCR fragments which contain known (non-repetitive) sequences; a multiplex polymerase chain reaction on the mixture (pool A) of synthesized PCR fragments with the aid of primers, which hybridize on the non-complementary linker sequences, and obtaining a mixture (pool B) with amplified sequence-controlled PCR fragments without repetitive portions, which are then, individually or in the mixture, suitable for a chromogenic or fluorescence in-situ hybridization (CISH or FISH) of chromosomes.

In one embodiment, the synthesized nucleic acid fragments which are present in the mixture after the first polymerase chain reaction are analyzed size-selectively and then purified. Furthermore, it is advantageous to add modified or labeled nucleotides (PCR labeling) in the last amplification step. If nucleotides which have been modified in the last amplification step are added, they can be of a type which allows chemical coupling with a chromogenic or fluorescent group, preferably aminoallyl NTPs.

In an alternative embodiment, the nucleic acid fragments which result from the first polymerase chain reaction are cloned in plasmids. The specialist will recognize that this can be done under restriction into the linker sequence. After amplification of the plasmids, the fragments can be generated in any quantity via the linker.

Probe fragments can also be subjected to a reaction which inserts or attaches reporter groups into/onto the hybridization probe. The labels inserted can be radioactive, chromophoric or fluorescent. The chromophoric group includes haptens such as biotin, avidin, digoxigenin, because these haptens can be made visible in an immunoreaction with a labeled antibody in the known way. Further chromophoric groups are enzymes such as peroxidases or lactases, which catalyze a color reaction. It is also possible to use modified nucleotides with a reactive group such as allylamine, which can be subjected to a reaction with appropriate groups of dyes.

The method disclosed has the advantage that the non-repetitive nucleic acid sequences selected in step (a) can be selected such that they are amplified in the first multiple polymerase chain reaction with essentially the same frequency. The sequence segments are preferably selected in step (a) such that non-repetitive PCR fragments with 100 to 5,000 base pairs, preferably with 100 to 1,000 base pairs, result. Particularly preferred are fragments with 400 to 600 base pairs. Furthermore, it is advantageous if the non-repetitive nucleic acid sequences which are selected in the analytical step are adjacent to one another on the genome under analysis so that a higher signal intensity results from the in-situ hybridization.

Several probes with different labeling are often required to detect chromosome aberrations. The disclosure therefore also encompasses the production of a large number of probes with different labels. It is advantageous if the specific nucleotide sequences selected in the first step are adjacent to a breakpoint region in the chromosome. Particularly advantageous and practical for diagnostic purposes is when the specific sequences selected in the analytical step flank a breakpoint region and have different labels, since a chromosome aberration can thus be diagnosed directly. The different labels of the probes can also be selected for adjacent sequences such that the color stains initially result in a compound color, and a color change or two different color signals can be observed in the case of an aberration. The reverse process can also take place, i.e. two different color signals can produce a compound signal or a fusion signal if there is an aberration. In other cases, it is also possible to select sequences which are from a single region, or which flank this region, which is amplified in the case of an aberration, if required as part of a balanced, unbalanced and reciprocal translocation.

The labeling of the probes is preferably selected from the group: chromogenic molecules, polymethine dyes, thiazole and oxazole dyes, Hoechst 33342 (2′-(4-ethoxyphenyl)-5-(4-methyl-1-piperazinyl)-2,5′-bi-1H-benzimidazole trihydrochloride), 4′,6-diamidin-2-phenylindole, Alexa 405, Alexa 488, Alexa 594, Alexa 633; Texas Red, rhodamine; sulfonated and non-sulfonated cyanine dyes, Cy2, Cy3, Cy5, Cy7; fluorescent molecules, fluorescein, 5,6-carbofluorescin, FITC (fluorescein isothiocyanate), GFP (Green Fluorescent Protein); chemiluminescent molecules, acridinium; ATTO®-fluorescent dyes (Atto-Tec, Siegen, DE), PromoFluor® dyes (PromoCell GmbH, Heidelberg, DE), MoBiTec® dyes (MoBiTec GmbH, Goettingen, DE), DY® dyes (DYOMICS GmbH, Jena, DE) Quantum Dots; haptens, digoxigenin, biotin, 2,4-dinitrophenol, avidin; enzymes for a chromogenic reaction, peroxidase, horseradish peroxidase, alkaline phosphatase.

One embodiment relates to the provision of a labeled probe for an in-situ hybridization to detect a chromosome aberration, comprising a plurality of PCR fragments whose sequences do not contain any repeats, pseudogenes or paralogous genes, and which are adjacent to each other on the human genome. A further embodiment relates to a probe mixture or a detection kit for a specific chromosome aberration which contains several differently labeled probes which flank the particular breakpoint regions.

Further advantages, embodiments and advantages of the invention are described in examples and with reference to the enclosed illustrations.

### BRIEF DESCRIPTION OF THE ILLUSTRATIONS

The following figures show:

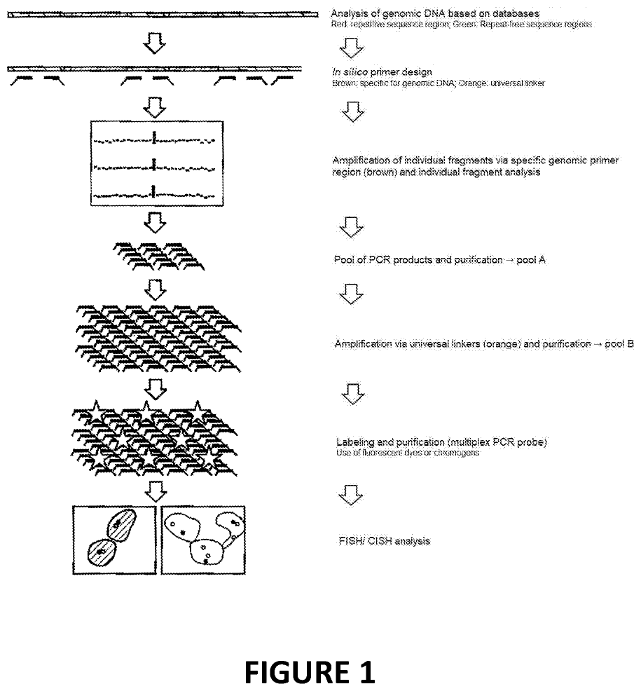

FIG. 1A diagram of the steps to produce sequence-controlled PCR probes for the in-situ hybridization (ISH) of chromosomes;

FIG. 2A schematic diagram of the steps to obtain repeat-reduced ISH probes according to the Prior Art;

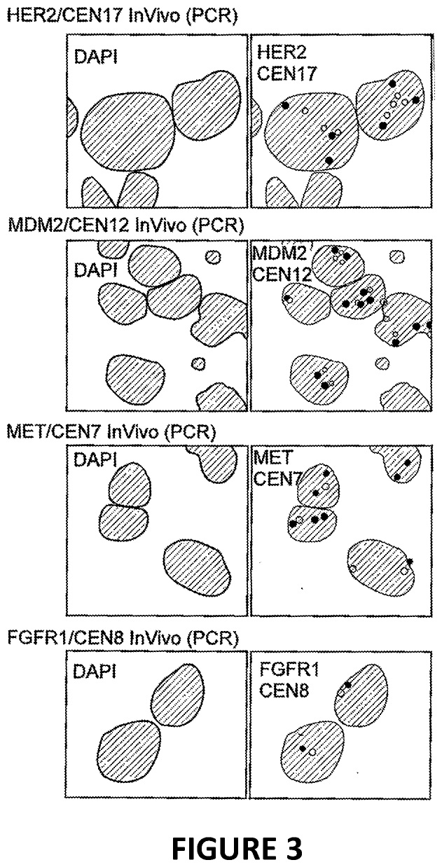

FIG. 3 Fluorescence microscopy images of samples after control staining of the cell nucleus with DAPI (4′,6-diaminophenolindol) and various in-situ hybridizations, where the sequence-controlled ISH probe was labeled in the PCR reaction (“one-step”): left column: control staining of the cell nucleus with DAPI; right column: in-situ hybridization with probes for the HER2, MDM2, MET and FGFR1 genes or the centromere;

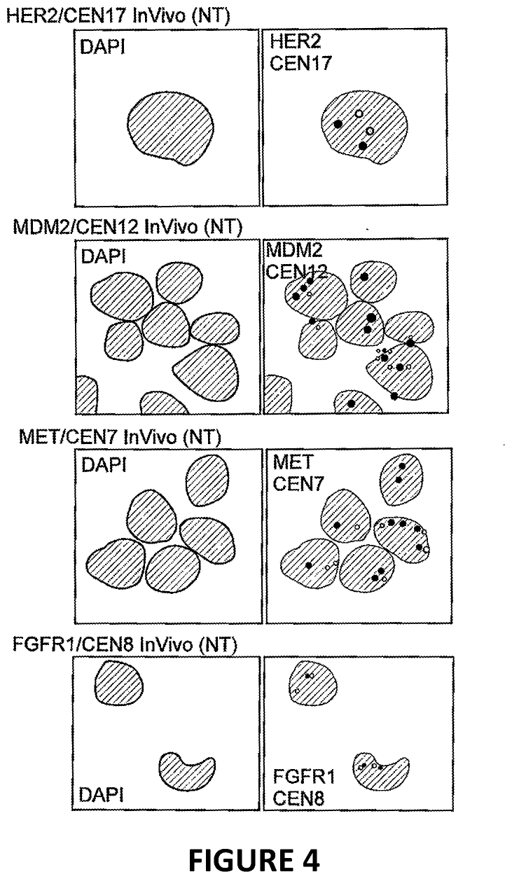

FIG. 4 Fluorescence microscopy images of samples after control staining of the cell nucleus with DAPI (4′,6-diaminophenolindol) and various in-situ hybridizations, where the sequence-controlled ISH probe was labeled by means of nick translation: left column: control staining of the cell nucleus with DAPI; right column: in-situ hybridization with probes for the HER2, MDM2, MET and FGFR1 genes or the centromere;

### DETAILED DESCRIPTION OF THE INVENTION

Sequence-controlled PCR probes can be generated from genomic DNA. The complete sequence of the human genome is known and can be obtained from databases. It is the starting point for the design of FISH/CISH probes. It is also possible to start with the sequence of BAC clones or other DNA carriers (plasmids, cosmids, fosmids, YACs) if the carriers and their sequences are available. Everything described below for genomic DNA can also be conducted with the other carriers.

See FIG. 1. In a first step (a), the genomic sequence and the genetic composition of the region is investigated by means of a computer analysis using the sequences in the databases. In this case, all regions with simple repeats, Alu sequences and complex repeats as well as all pseudogenes and paralogous sections are sought and identified in the genomic sequence. These sequences are unsuitable for the ISH probe. The NCBI (National Center for Biotechnology Information, Bethesda, Maryland) or ENSEMBL (maintained by the European Bioinformatics Institute (EBI) and the European Molecular Biology Laboratory (EMBL), Heidelberg, DE) provide public genome databases or they can be accessed via the UCSC genome browser (University of California, Santa Cruz, US). Any nonspecific sequence regions can be further narrowed down and identified with the aid of conventional analytical programs.

Only sequences which are specific to a locus and occur once are included in the hybridization probe. Excluded are sequences which hybridize at other loci also during a chromosome hybridization and thus contribute nonspecifically to the background (step a). For the genomic sequences controlled in this way, which occur only once in the chromosome, a library of sense and antisense primers is then collated (b). Each primer is synthesized with a specific primer sequence and a uniform linker. Sense and antisense primers are planned and selected on the basis of the known genome sequence so that the PCR and the subsequent amplification result in DNA fragments of essentially the same length. Typically, 200 primers are designed and synthesized for every genome region so that, after the PCR, the locus on the genome is covered section-by-section by 100 DNA fragments. In a first amplification, the specific fragments are generated in individual PCR reactions with the aid of the primers; the resulting PCR fragments are checked for purity and size and combined or pooled (pool A) in step (d). After purification of the pool (pool A), 10 ng of pooled PCR-DNA is used as the matrix for a second amplification—(step e)—where the linkers serve as sense and antisense primers in this amplification. Since all the fragments of the A-pool have the same linkers and a planned similar size, all the PCR fragments from pool A are amplified to the same extent in this reaction (multiplex PCR). The result is a mixed pool of PCR fragments (pool B).

If labeled nucleotides are added in this last reaction (one-step PCR labeling), the result is a pool B with labeled PCR fragments. After purification, these can be used in the in-situ hybridization. Alternatively, pool B containing the mixed PCR fragments can also be labeled by nick translation after the second amplification.

In order to detect a gene locus robustly in the FISH/CISH, it is usually necessary to investigate genomic regions of 100 to 1,000 kilobases for the probe design. This means that 1 to 10 multiplex PCR probes are produced, which in combination make up the actual probe. The probe thus produced contains no repetitive elements, pseudogenes, or paralogs from the very start of the production. Any quantity of the probe can be produced. Since the individual PCR fragments are essentially of similar size, or their length is specified, the direct insertion of labeled nucleotides is no longer a very demanding procedure. In the usual multiplex PCR or linker PCR, the following can be used: (a) non-chemically modified or unmodified dNTPs when the subsequent labeling is done by conventional enzymatic methods such as nick translation, (b) dNTPs labeled with fluorescent dyes (for example Atto488 or Cy3) or with haptens (for example digoxigenin, dinitrophenol or biotin), where the labeling can be done directly in the linker PCR. In respect of the addition of the dNTPs labeled with fluorescent dye or hapten, it is necessary to determine the ratio in which they are added. Depending on labeling and dNTP (dUTP: fluorescence-labeled dNTP), this ratio will be between 1:1 and 1:20; or (c) chemically reactive dNTPs, such as aminoallyl dNTP. The labeling can also be done after the synthesis by means of chemical coupling with amine-reactive NHS ester-activated (NHS=N-hydroxysuccinimide) dye or hapten derivates. Reactive aminoallyl nucleotides or aminoallyl dNTPs are added in a ratio of between 5:1 and 1:5, depending on the probe type (gene probe or centromere probe). The DNA probe is then labeled post-synthesis by a reaction with amine-reactive dye or fluorescent dye. If the DNA probe is not labeled directly with fluorescent dyes or haptens, the labeling must be done after the linker amplification.

### EXAMPLES

### Example 1

### Production of a Locus Probe for the HER2 Gene

### 1. Design of the HER2 Locus Probe in Silico

On chromosome 17, the genome region with the HER2 gene and large 5′ and 3′ flanks was selected for the design of the HER2 (ERBB2) gene probe. The HER2 gene probe covered the section from 39,395,605 to 39,799,506 on chromosome 17. The genome sequence was analyzed in the Ensembl (www.ensembl.org) and NCBI (https://www.ncbi.nlm.nih.gov/) databases, and four suitable sections were identified for the probe. The first genome section was chromosome 17: 39,395,605-39,569,361. The explanation below refers only to this section; the three other genomic sections at the HER2 locus were processed analogously.

All repetitive elements, Alu sequences, pseudogenes and paralogous sequence sections in the genome section 39,395,605-39,569,361 were identified with conventional analytical programs. Repetitive elements, pseudogenes and paralogous sequences were excluded. The sequences specific to the first genome section were split up into sections with 250 to 800 base pairs. Partial sequences with less than 250 base pairs were not taken into account. After this analysis, the target genomic sequence in each of the four selected genomic sections contained only 40 to 60 percent of the starting sequence—approx. half of the genomic starting sequence was thus rejected as unspecific.

### 2. In Silico Primer Design

Sense and antisense primers for the specific sequence sections were planned and synthesized for genome fragments with 250 to 800 base pairs. On the one hand, the primers were complementary to the genome sequence which was to be amplified; on the other hand, they also contained a universal linker sequence with a cleavage site for a restriction endonuclease. Sense and antisense primers were planned such that the resulting products in the PCR on the genome were of similar length. The desired fragment length was approx. 500 base pairs. The only deviation from this was when the specificity or the functionality of the primer pair required it. Approx. 200 primers were designed and synthesized for each of the four genomic sections. Table 1, which is appended to the description, contains a representative list of the thus determined sense and antisense primers for the genome section 39,395,605-39,569.361 on chromosome 17.

### 3. Amplification of the Individual Fragments

The genome fragments were amplified in 50 μl solutions for every sense and antisense primer pair. The individual PCR reactions were conducted in the high-throughput method (96-well) at an attachment temperature (primer annealing) of 55° C. and 15 seconds strand elongation over 35 cycles in each case. The PCR fragments obtained were checked for purity and size on the agarose gel. Only PCR products with a specific band of expected size were used so that their sequence was effectively controlled and known. The yield of correct PCR products was over 90%.

4. Cutting of the Individual Fragments with Restriction Enzymes and Cloning

The PCR products were usually blunt cloned into a carrier plasmid without cutting and thus secured. Some PCR products were also cloned into a plasmid after cutting. The occasional check of the sequence correctness was unproblematic and conducted in the known way.

### 5. Pooling and Processing of the Individual Fragments

The approx. 100 PCR products were pooled and processed to remove primers, proteins, free nucleotides and salts. A photometric measurement and optical analysis of the mixture (pool A) by agarose gel electrophoresis followed.

### 6. Amplification of the Pool via Universal Linkers (Multiplex PCR) and Labeling

The second amplification via the universal linkers was done with a high-performance Taq polymerase for large yields, directly with the mixture of the individual fragments (pool A) as the template (multiplex PCR). dNTPs labeled with fluorescent dyes (e.g. Atto488 or Cy3) were used in this multiplex PCR/linker PCR. The fluorescence-labeled dNTPs were added in a tested ratio which was dependent on the labeled dNTP and differed according to the type of probe (gene probe or centromere probe). The ratio was between 5:1 and 1:5 across the different probes and depending on the labeling.

### 7. Purification of the Labeled DNA Probe

The labeling and amplification reaction was followed by a final purification of the labeled multiplex PCR probes by precipitation and chromatography, where free dNTPs, labeled nucleotides, sense and antisense primers as well as the enzyme were removed. This was followed by a photometric measurement and determination of the insertion rate of the fluorescence in the FISH probes as well as a final check of the probe by agarose gel electrophoresis.

### 8. Additional Fragmentation of DNA Probes

As a further option, the DNA probes were post-fragmented if they produced too much background in the ISH because of their length. In general, a length of 200 to 300 base pairs is favorable for ISH. The post-fragmentation was done physically, but can also be done enzymatically or chemically. The fragment size distribution was analyzed by agarose gel electrophoresis.

### 9. Completion of the DNA Probe

The HER2 locus probe comprised four segments. The above-mentioned steps 1 to 8 concerned a first section. The final DNA probe consisted of four separate preparations (4 multiplex PCR probes). Alternatively, these four individual preparations can be combined after the universal linker amplification, and labeled and purified together, although this affords less control. Depending on locus, probe type (amplification probe, break-apart probe, fusion probe) and user specifications, a DNA probe mixture will consist of 1 to 10 preparations (multiplex PCR probes).

### Example 2

### In-Situ Hybridization of Chromosomes with Labeled PCR Probes

Version 1: ISH with DNA probes can be conducted using standard methods on tissues or cells, for example in accordance with the recommendations of the Laboratory Working Group of the DGHO (German Society for Hematology and Medical Oncology). In this case, in-situ hybridization takes place on interphase cells of cell cultures or tissues. The target DNA in each case is the nuclear DNA of interphase cell nuclei, which are fixed on a specimen slide. The probe here is produced and labeled as described in Example 1. The cell nuclei are typically counterstained with the fluorochrome DAPI (4′,6-diamidino-2-phenylindole). In principal, FISH can be conducted on the following materials: peripheral blood (PB), bone marrow (KM), paraffin sections, tumor tissue, cytospin preparations, amniotic fluid, cells/metaphases fixed with methanol glacial acetic acid, etc. Other patient samples such as blood or bone marrow are fixed with methanol/ethanoic acid (ratio 3:1) after Ficoll separation and frozen at −20° C. until hybridization. Special pretreatments are necessary for amniotic fluid or paraffin sections.

Version 2: In the examples (see FIGS. 3 and 4), the cells of cell lines (cell cultures) embedded in paraffin were cut on the microtome according to a standard method and drawn onto a cleaned specimen slide. The cells embedded in paraffin were subsequently treated according to standard methods also. Deparaffinization was ensured by means of a series of graded washes of xylene and an alcohol series. A pretreatment was carried out at 95° C. for only a few minutes in a citrate pretreatment buffer. A partial digest of the cells with pepsin solution followed in order to free the cell nuclei as much as possible from cytoplasm and make them accessible. After dehydration in serial alcohol washes, a probe-hybridization mixture was pipetted onto the cells and sealed with a cover glass and sealant. The cell nuclei and the hybridization sample were denatured at 75° C. for 10 minutes, slowly cooled to 37° C. and hybridized in a humid chamber at 37° C. overnight (16 hrs). After the cover glass had been removed, there followed two rinses with rinsing solution at 37° C. The hybridization signals were evaluated and documented on the fluorescence microscope with appropriate filters. FIGS. 3 and 4 depict the results for differently labeled probes (by nick translation or one-step-PCR labeling). In both cases, the hybridization signals were clearly visible and the background hybridization was negligible.

TABLE 1
Sense and antisense primers for the
genome section 39,395,605-39,569,361
on chromosome 17.

| | | | DNA to be |
|---|---|---|---|
| | | | amplified |
| | | Primer sequence | in base |
| SEQ ID: | Name | 5′ to 3′ | pairs |
| SEQ ID | D2_s_254 | TGTAAAACGACGGCC | |
| NO: 001 | | AGTCTTAGTTTCACC | |
| | | AGTACTCAATCCC | |
| SEQ ID | D2_as_254 | CAGGAAACAGCTATG | 522 |
| NO: 002 | | ACCCTCAGTCTCAGA | |
| | | ATACACCGTTAAG | |
| SEQ ID | D2_s_255 | TGTAAAACGACGGCC | |
| NO: 003 | | AGTCTAGGGTTACAG | |
| | | GTTGCACATAATA | |
| SEQ ID | D2_as_255 | CAGGAAACAGCTATG | 409 |
| NO: 004 | | ACCGCCTTCATTTTA | |
| | | AGTGCCATAACTG | |
| SEQ ID | D2_s_256 | TGTAAAACGACGGCC | |
| NO: 005 | | AGTTGGCCTCCAGTT | |
| | | ACAACATATAAAA | |
| SEQ ID | D2_as_256 | CAGGAAACAGCTATG | 723 |
| NO: 006 | | ACCCTCTGTAGGGGT | |
| | | GGCATACTATAAT | |
| SEQ ID | D2_s_257 | TGTAAAACGACGGCC | |
| NO: 007 | | AGTCCCGACTCAAAT | |
| | | CTTCAGTATCTG | |
| SEQ ID | D2_as_257 | CAGGAAACAGCTATG | 425 |
| NO: 008 | | ACCAGGGTGTGAAAA | |
| | | CTTTTAAGGGTT | |
| SEQ ID | D2_s_258 | TGTAAAACGACGGCC | |
| NO: 009 | | AGTAAAGACCAGGTG | |
| | | ACATTTACTCTTC | |
| SEQ ID | D2_as_258 | CAGGAAACAGCTATG | 456 |
| NO: 010 | | ACCGAGTTTGAGACC | |
| | | TTGGTGAAACTAG | |
| SEQ ID | D2_s_259 | TGTAAAACGACGGCC | |
| NO: 011 | | AGTAACAAACGCGTC | |
| | | ATGAAGGTAG | |
| SEQ ID | D2_as_259 | CAGGAAACAGCTATG | 405 |
| NO: 012 | | ACCAAGAGCAGGTTT | |
| | | GAGGTGTGAG | |
| SEQ ID | D2_s_260 | TGTAAAACGACGGCC | |
| NO: 013 | | AGTCAGTGCATTACA | |
| | | TTACGGGGTG | |
| SEQ ID | D2_as_260 | CAGGAAACAGCTATG | 744 |
| NO: 014 | | ACCAAGTCAGTCGCT | |
| | | AAAACCACTAATT | |
| SEQ ID | D2_s_261 | TGTAAAACGACGGCC | |
| NO: 015 | | AGTTGGCCTACAGAG | |
| | | TAATAGAATGAAG | |
| SEQ ID | D2_as_261 | CAGGAAACAGCTATG | 503 |
| NO: 016 | | ACCCCCCATACATTT | |
| | | CTATCCTTGAGT | |
| SEQ ID | D2_s_262 | TGTAAAACGACGGCC | |
| NO: 017 | | AGTTGAGAATTCATG | |
| | | ATATTGCTGCACT | |
| SEQ ID | D2_as_262 | CAGGAAACAGCTATG | 534 |
| NO: 018 | | ACCGATAAGCCCCTG | |
| | | AATTGTCGATG | |
| SEQ ID | D2_s_263 | TGTAAAACGACGGCC | |
| NO: 019 | | AGTTTCTTTGATCTG | |
| | | GGATGAAGACAG | |
| SEQ ID | D2_as_263 | CAGGAAACAGCTATG | 526 |
| NO: 020 | | ACCAGGTTGGAGTCA | |
| | | GCATAGAGTTG | |
| SEQ ID | D2_s_264 | TGTAAAACGACGGCC | |
| NO: 021 | | AGTATATCAAAGTAA | |
| | | GGCCGCAGAATTT | |
| SEQ ID | D2_as_264 | CAGGAAACAGCTATG | 524 |
| NO: 022 | | ACCAAACATGTTTTC | |
| | | AGATGCAGTAAGG | |
| SEQ ID | D2_s_265 | TGTAAAACGACGGCC | |
| NO: 023 | | AGTGATTACAAGTCA | |
| | | ATACCTCCACAGA | |
| SEQ ID | D2_as_265 | CAGGAAACAGCTATG | 504 |
| NO: 024 | | ACCATGTATATGAAG | |
| | | GGCTGGGCAG | |
| SEQ ID | D2_s_266 | TGTAAAACGACGGCC | |
| NO: 025 | | AGTAGTATTTTAACT | |
| | | TTCTTTCTGGCACA | |
| SEQ ID | D2_as_266 | CAGGAAACAGCTATG | 356 |
| NO: 026 | | ACCGACAAGTAACAC | |
| | | AATCTTATCTGCA | |
| SEQ ID | D2_s_267 | TGTAAAACGACGGCC | |
| NO: 027 | | AGTAGCTGGGACTAT | |
| | | TCTGATAAGATTT | |
| SEQ ID | D2_as_267 | CAGGAAACAGCTATG | 530 |
| NO: 028 | | ACCTGTCATAGATAA | |
| | | ACTGAAGCATGGG | |
| SEQ ID | D2_s_268 | TGTAAAACGACGGCC | |
| NO: 029 | | AGTGTTCTGAGAGGA | |
| | | TGACATGGAAG | |
| SEQ ID | D2_as_268 | CAGGAAACAGCTATG | 503 |
| NO: 030 | | ACCAATCAGAAGGTT | |
| | | CATCAAGTTCCAA | |
| SEQ ID | D2_s_269 | TGTAAAACGACGGCC | |
| NO: 031 | | AGTCTGGTATACTGA | |
| | | CTGTGAGAGGAAG | |
| SEQ ID | D2_as_269 | CAGGAAACAGCTATG | 500 |
| NO: 032 | | ACCAAGTTTAAGGGC | |
| | | AATAACCAAGCC | |
| SEQ ID | D2_s_27O | TGTAAAACGACGGCC | |
| NO: 033 | | AGTCCACTTTGGCTG | |
| | | CTTTCATCAAA | |
| SEQ ID | D2_as_270 | CAGGAAACAGCTATG | 602 |
| NO: 034 | | ACCAAGAAGTCATCA | |
| | | AGATTACCACCTG | |
| SEQ ID | D2_s_271 | TGTAAAACGACGGCC | |
| NO: 035 | | AGTTTTCTAAAGGGC | |
| | | TGCTTCCATAAA | |
| SEQ ID | D2_as_271 | CAGGAAACAGCTATG | 720 |
| NO: 036 | | ACCTTGTGTGATTCA | |
| | | TAAGAGGTTGACA | |
| SEQ ID | D2_s_272 | TGTAAAACGACGGCC | |
| NO: 037 | | AGTACATCTCTAAAT | |
| | | GGGACTCAGATGA | |
| SEQ ID | D2_as_272 | CAGGAAACAGCTATG | 363 |
| NO: 038 | | ACCCTTTTAGATTCT | |
| | | CCTGGGCTTCTC | |
| SEQ ID | D2_s_273 | TGTAAAACGACGGCC | |
| NO: 039 | | AGTGACTGAAATTCT | |
| | | CTCTCCCTCCTT | |
| SEQ ID | D2_as_273 | CAGGAAACAGCTATG | 614 |
| NO: 040 | | ACCAGTAGTTGGAGT | |
| | | AATAACATAGAGCA | |
| SEQ ID | D2_s_274 | TGTAAAACGACGGCC | |
| NO: 041 | | AGTATAGTTCTTTTG | |
| | | ACACAGCTTCCAA | |
| SEQ ID | D2_as_274 | CAGGAAACAGCTATG | 411 |
| NO: 042 | | ACCTGTTAGCTAGGT | |
| | | TAGGAAATCACTA | |
| SEQ ID | D2_s_275 | TGTAAAACGACGGCC | |
| NO: 043 | | AGTGCAGTCTTGAAG | |
| | | AAACTTATCTGAC | |
| SEQ ID | D2_as_275 | CAGGAAACAGCTATG | 646 |
| NO: 044 | | ACCATATCAAGTCAG | |
| | | AATCCAACCCTTC | |
| SEQ ID | D2_s_276 | TGTAAAACGACGGCC | |
| NO: 045 | | AGTGTACAAACTCTC | |
| | | AAAAGCTCACTCT | |
| SEQ ID | D2_as_276 | CAGGAAACAGCTATG | 521 |
| NO: 046 | | ACCCTATCCCCAGTT | |
| | | TCTCGATCTTTG | |
| SEQ ID | D2_s_277 | TGTAAAACGACGGCC | |
| NO: 047 | | AGTCATAATGGTTCT | |
| | | GTCTTGTAATTGGG | |
| SEQ ID | D2_as_277 | CAGGAAACAGCTATG | 443 |
| NO: 048 | | ACCATGTTCTTCTCA | |
| | | TCTACTCTCCAGC | |
| SEQ ID | D2_s_278 | TGTAAAACGACGGCC | |
| NO: 049 | | AGTTGACTGGCTTCA | |
| | | CTACTATTAAAGC | |
| SEQ ID | D2_as_278 | CAGGAAACAGCTATG | 606 |
| NO: 050 | | ACCTTGAGGAAAATG | |
| | | GTTAAAGCTGATT | |
| SEQ ID | D2_s_279 | TGTAAAACGACGGCC | |
| NO: 051 | | AGTCAACACTTGTGG | |
| | | GTATACAGCA | |
| SEQ ID | D2_as_279 | CAGGAAACAGCTATG | 431 |
| NO: 052 | | ACCTATTTTGCTGCT | |
| | | ACTCGTTTCTTTT | |
| SEQ ID | D2_s_280 | TGTAAAACGACGGCC | |
| NO: 053 | | AGTGATAGAGTAGGG | |
| | | TGAAAAGCTATGC | |
| SEQ ID | D2_as_280 | CAGGAAACAGCTATG | 419 |
| NO: 054 | | ACCTGCTTTGACTTT | |
| | | AAATTTCTCACCC | |
| SEQ ID | D2_s_281 | TGTAAAACGACGGCC | |
| NO: 055 | | AGTCTTGTTAAAGCC | |
| | | TTTGGATGATGTC | |
| SEQ ID | D2_as_281 | CAGGAAACAGCTATG | 658 |
| NO: 056 | | ACCCAGCTAACCCTA | |
| | | ATCAGTGAACTTT | |
| SEQ ID | D2_s_282 | TGTAAAACGACGGCC | |
| NO: 057 | | AGTTCCCCAGGTATC | |
| | | TTATATTACCCTT | |
| SEQ ID | D2_as_282 | CAGGAAACAGCTATG | 522 |
| NO: 058 | | ACCTATATGGATTTT | |
| | | GCATGGATGGACA | |
| SEQ ID | D2_s_283 | TGTAAAACGACGGCC | |
| NO: 059 | | AGTCTCCTAAAGTTG | |
| | | AAACCACCATGTA | |
| SEQ ID | D2_as_283 | CAGGAAACAGCTATG | 421 |
| NO: 060 | | ACCTAACTTGTGTGT | |
| | | CATCTTGCCATTA | |
| SEQ ID | D2_s_284 | TGTAAAACGACGGCC | |
| NO: 061 | | AGTAGAACTATGCCC | |
| | | AAAATGCTAAGG | |
| SEQ ID | D2_as_284 | CAGGAAACAGCTATG | 451 |
| NO: 062 | | ACCTTTAGATTGTCA | |
| | | CTATACCCTCCTT | |
| SEQ ID | D2_s_285 | TGTAAAACGACGGCC | |
| NO: 063 | | AGTCTCCTTTCCCCT | |
| | | ACAGTCACTTA | |
| SEQ ID | D2_as_285 | CAGGAAACAGCTATG | 506 |
| NO: 064 | | ACCATCTGGAGATTT | |
| | | TAATTTGCGAGCT | |
| SEQ ID | D2_s_286 | TGTAAAACGACGGCC | |
| NO: 065 | | AGTGTGAAACGGAGG | |
| | | CAAGAAAGGG | |
| SEQ ID | D2_as_286 | CAGGAAACAGCTATG | 319 |
| NO: 066 | | ACCGCGAGGGAAGAA | |
| | | ATGAGAGTTC | |
| SEQ ID | D2_s_287 | TGTAAAACGACGGCC | |
| NO: 067 | | AGTCTTCTTCAGGTC | |
| | | AGGGGAAAGG | |
| SEQ ID | D2_as_287 | CAGGAAACAGCTATG | 507 |
| NO: 068 | | ACCATAGTCATCAGT | |
| | | CTCCTCATTCGAA | |
| SEQ ID | D2_s_288 | TGTAAAACGACGGCC | |
| NO: 069 | | AGTACAAAGACCGGA | |
| | | GTAAAAGTCATC | |
| SEQ ID | D2_as_288 | CAGGAAACAGCTATG | 545 |
| NO: 070 | | ACCCAGACAATGTTA | |
| | | ACACGCTGAATG | |
| SEQ ID | D2_s_289 | TGTAAAACGACGGCC | |
| NO: 071 | | AGTCAGAATTCGAGA | |
| | | AGTGAAGAGTACA | |
| SEQ ID | D2_as_289 | CAGGAAACAGCTATG | 589 |
| NO: 072 | | ACCACAAAATTTCAA | |
| | | ACTAGCATGCCTT | |
| SEQ ID | D2_s_290 | TGTAAAACGACGGCC | |
| NO: 073 | | AGTAACTTAAAATGA | |
| | | TCTGCCCCATAGA | |
| SEQ ID | D2_as_290 | TGTAAAACGACGGCC | 451 |
| NO: 074 | | AGTAACTTAAAATGA | |
| | | TCTGCCCCATAGA | |
| SEQ ID | D2_s_291 | TGTAAAACGACGGCC | |
| NO: 075 | | AGTAACTTAAAATGA | |
| | | TCTGCCCCATAGA | |
| SEQ ID | D2_as_291 | CAGGAAACAGCTATG | 411 |
| NO: 076 | | ACCCCATAGTAAAGG | |
| | | TGGGTTATATCCA | |
| SEQ ID | D2_s_292 | TGTAAAACGACGGCC | |
| NO: 077 | | AGTAGTCCATTCATT | |
| | | TAAAACTGGCTTT | |
| SEQ ID | D2_as_292 | CAGGAAACAGCTATG | 491 |
| NO: 078 | | ACCTGTTCCCTGTGC | |
| | | TTTCAAATCTTTA | |
| SEQ ID | D2_s_293 | TGTAAAACGACGGCC | |
| NO: 079 | | AGTCCAAAGGAGACA | |
| | | GAAACATCAGAA | |
| SEQ ID | D2_as_293 | CAGGAAACAGCTATG | 578 |
| NO: 080 | | ACCGTTAACCAGGCC | |
| | | TCAAATTCTAATT | |
| SEQ ID | D2_s_294 | TGTAAAACGACGGCC | |
| NO: 081 | | AGTCCACAGATTCAG | |
| | | TTCAGATTCCAAA | |
| SEQ ID | D2_as_294 | CAGGAAACAGCTATG | 430 |
| NO: 082 | | ACCGGTTATGTCCTA | |
| | | CCAGTTCAGGATA | |
| SEQ ID | D2_s_295 | TGTAAAACGACGGCC | |
| NO: 083 | | AGTGCATGTTCATCT | |
| | | CTCCCAGTATTTC | |
| SEQ ID | D2_as_295 | CAGGAAACAGCTATG | 462 |
| NO: 084 | | ACCTAGTACAACAGA | |
| | | CTAACTCATGTCT | |
| SEQ ID | D2_s_296 | TGTAAAACGACGGCC | |
| NO: 085 | | AGTTCTACAAAACCC | |
| | | CACTGACTACTAT | |
| SEQ ID | D2_as_296 | CAGGAAACAGCTATG | 426 |
| NO: 086 | | ACCAATTCACAAAGT | |
| | | ATCACTGCAACTC | |
| SEQ ID | D2_s_297 | TGTAAAACGACGGCC | |
| NO: 087 | | AGTTAACCTTTTCGT | |
| | | AACCAGGCATTAT | |
| SEQ ID | D2_as_297 | CAGGAAACAGCTATG | 605 |
| NO: 088 | | ACCACACGATTGACA | |
| | | CATCCTGAATTTA | |
| SEQ ID | D2_s_298 | TGTAAAACGACGGCC | |
| NO: 089 | | AGTGACTTAATGGGA | |
| | | CTGCTAGAATCTG | |
| SEQ ID | D2_as_298 | CAGGAAACAGCTATG | 534 |
| NO: 090 | | ACCTCTGATATTACC | |
| | | AATGCAGTATAGGC | |
| SEQ ID | D2_s_299 | TGTAAAACGACGGCC | |
| NO: 091 | | AGTGCTTTCTCTGAC | |
| | | TCATAATTCCCAG | |
| SEQ ID | D2_as_299 | CAGGAAACAGCTATG | 560 |
| NO: 092 | | ACCCGGCATAAACTG | |
| | | ATAATTTGGTGAC | |
| SEQ ID | D2_s_300 | TGTAAAACGACGGCC | |
| NO: 093 | | AGTGCCCTCTTAAGT | |
| | | CTTTCTCAATCTAG | |
| SEQ ID | D2_as_300 | CAGGAAACAGCTATG | 468 |
| NO: 094 | | ACCTCTTCAGAGTTA | |
| | | TAGAGCCGAGC | |
| SEQ ID | D2_s_301 | TGTAAAACGACGGCC | |
| NO: 095 | | AGTTCTTCATCTCCT | |
| | | ACTTCTGAACAGT | |
| SEQ ID | D2_as_301 | CAGGAAACAGCTATG | 407 |
| NO: 096 | | ACCGGAAATCTGGCC | |
| | | AAAAGTTTATCG | |
| SEQ ID | D2_s_302 | TGTAAAACGACGGCC | |
| NO: 097 | | AGTAAAAGACTCTTG | |
| | | ACTCCCTTTCTCT | |
| SEQ ID | D2_as_302 | CAGGAAACAGCTATG | 616 |
| NO: 098 | | ACCCATCTGAGCTAT | |
| | | GAATGGAAGAGC | |
| SEQ ID | D2_s_3O3 | TGTAAAACGACGGCC | |
| NO: 099 | | AGTTCTTCTCATCTC | |
| | | ACAGTTTACCCTA | |
| SEQ ID | D2_as_303 | CAGGAAACAGCTATG | 613 |
| NO: 100 | | ACCCTACACTGTGTT | |
| | | GCCATGATAAACT | |
| SEQ ID | D2_s_304 | TGTAAAACGACGGCC | |
| NO: 101 | | AGTGATGAGCAAGTT | |
| | | GATATCCAGTCAT | |
| SEQ ID | D2_as_304 | CAGGAAACAGCTATG | 532 |
| NO: 102 | | ACCAAAGGGGAAATA | |
| | | TGTAAGGGAATGC | |
| SEQ ID | D2_s_305 | TGTAAAACGACGGCC | |
| NO: 103 | | AGTCTCCATCCAAAA | |
| | | CTTCTCGAAAAGA | |
| SEQ ID | D2_as_305 | CAGGAAACAGCTATG | 636 |
| NO: 104 | | ACCTTTTGTCCCGAA | |
| | | GAATTGGTCATAA | |
| SEQ ID | D2_s_306 | TGTAAAACGACGGCC | |
| NO: 105 | | AGTGAAGTCACTTTT | |
| | | ATGCACAATCCAG | |
| SEQ ID | D2_as_306 | CAGGAAACAGCTATG | 565 |
| NO: 106 | | ACCAGAACTGCCAAT | |
| | | ATATTCTGCATGT | |
| SEQ ID | D2_s_307 | TGTAAAACGACGGCC | |
| NO: 107 | | AGTCTGATGAAAACC | |
| | | CAAGAGCCAG | |
| SEQ ID | D2_as_307 | CAGGAAACAGCTATG | 494 |
| NO: 108 | | ACCGTCTGATAATGG | |
| | | TTTGTTTGGAAGT | |
| SEQ ID | D2_s_308 | TGTAAAACGACGGCC | |
| NO: 109 | | AGTCCTGGAAGATAC | |
| | | AATCAAGAAAACC | |
| SEQ ID | D2_as_308 | CAGGAAACAGCTATG | 489 |
| NO: 110 | | ACCCATATACTTTCT | |
| | | GCACCACCTCATA | |
| SEQ ID | D2_s_309 | TGTAAAACGACGGCC | |
| NO: 111 | | AGTGACCTCAAGTGT | |
| | | TAGTTCTGTTGAA | |
| SEQ ID | D2_as_309 | CAGGAAACAGCTATG | 418 |
| NO: 112 | | ACCTGTATAGTACCC | |
| | | ACCCAAGTTATTT | |
| SEQ ID | D2_s_310 | TGTAAAACGACGGCC | |
| NO: 113 | | AGTCAGCAGGATGAC | |
| | | TCTCAATTTTCT | |
| SEQ ID | D2_as_310 | CAGGAAACAGCTATG | 504 |
| NO: 114 | | ACCTTCTCCAAACTT | |
| | | TAGTCACGTCTC | |
| SEQ ID | D2_s_311 | TGTAAAACGACGGCC | |
| NO: 115 | | AGTGTAAAATGTGTT | |
| | | AGCCAAGTCCATG | |
| SEQ ID | D2_as_311 | CAGGAAACAGCTATG | 423 |
| NO: 116 | | ACCGCACATGTGATT | |
| | | ATAAACACAAACA | |
| SEQ ID | D2_s_312 | TGTAAAACGACGGCC | |
| NO: 117 | | AGTGCAACTTTTATC | |
| | | CCAGCCTGAAG | |
| SEQ ID | D2_as_312 | CAGGAAACAGCTATG | 582 |
| NO: 118 | | ACCCTGAAGTCTCTG | |
| | | GGTTAGTAAGGAA | |
| SEQ ID | D2_s_313 | TGTAAAACGACGGCC | |
| NO: 119 | | AGTGTATTCCTTGCT | |
| | | CACTTGCTACTAG | |
| SEQ ID | D2_as_313 | CAGGAAACAGCTATG | 530 |
| NO: 120 | | ACCAGTGGAGTAAAC | |
| | | TTTAAGGATGAGT | |
| SEQ ID | D2_s_314 | TGTAAAACGACGGCC | |
| NO: 121 | | AGTCTCTTTGAGGCA | |
| | | TTTGTTTGGAAAA | |
| SEQ ID | D2_as_314 | CAGGAAACAGCTATG | 681 |
| NO: 122 | | ACCACTCACATAGAA | |
| | | ATAGAAGCCAGAA | |
| SEQ ID | D2_s_315 | TGTAAAACGACGGCC | |
| NO: 123 | | AGTGACTGTTTTCCC | |
| | | CTGTTAATGAGAT | |
| SEQ ID | D2_as_315 | CAGGAAACAGCTATG | 443 |
| NO: 124 | | ACCATTGATGACTGC | |
| | | TGATAACCAAAAT | |
| SEQ ID | D2_s_316 | TGTAAAACGACGGCC | |
| NO: 125 | | AGTATATACTTAAGC | |
| | | CTCCATTCCCTCA | |
| SEQ ID | D2_as_316 | CAGGAAACAGCTATG | 527 |
| NO: 126 | | ACCTTATCGTTCCTA | |
| | | TGGACAGTTGAAG | |
| SEQ ID | D2_s_317 | TGTAAAACGACGGCC | |
| NO: 127 | | AGTTGATTTAGGTTC | |
| | | CTGACACTGATTC | |
| SEQ ID | D2_as_317 | CAGGAAACAGCTATG | 648 |
| NO: 128 | | ACCAATCTCAGCAAA | |
| | | GGTAGGTACACTA | |
| SEQ ID | D2_s_318 | TGTAAAACGACGGCC | |
| NO: 129 | | AGTTTGTTTCCAAAA | |
| | | TCTTTCCCATCTC | |
| SEQ ID | D2_as_318 | CAGGAAACAGCTATG | 542 |
| NO: 130 | | ACCAATCTAGGTTAG | |
| | | TAAGGTCCTCAGC | |
| SEQ ID | D2_s_319 | TGTAAAACGACGGCC | |
| NO: 131 | | AGTTTCAGTACCTCA | |
| | | ATTTCTTAGGCAC | |
| SEQ ID | D2_as_319 | CAGGAAACAGCTATG | 687 |
| NO: 132 | | ACCGTAGGTTGAGGA | |
| | | TCCAGATAGACAT | |
| SEQ ID | D2_s_320 | TGTAAAACGACGGCC | |
| NO: 133 | | AGTGCTTTTCATTTG | |
| | | TGTTATGTAGGGTAC | |
| SEQ ID | D2_as_320 | CAGGAAACAGCTATG | 528 |
| NO: 134 | | ACCAAAGTCCATCCA | |
| | | TTTACAGGTCCTA | |
| SEQ ID | D2_s_321 | TGTAAAACGACGGCC | |
| NO: 135 | | AGTTAAGAAGTCCAC | |
| | | CATATAGCAGTCA | |
| SEQ ID | D2_as_321 | CAGGAAACAGCTATG | 416 |
| NO: 136 | | ACCGTTCTCCCTTCT | |
| | | TACCCTTATTTCC | |
| SEQ ID | D2_s_322 | TGTAAAACGACGGCC | |
| NO: 137 | | AGTAGATTCACAGTT | |
| | | CATTTCAGGAAGA | |
| SEQ ID | D2_as_322 | CAGGAAACAGCTATG | 365 |
| NO: 138 | | ACCAAAATGAGTGGC | |
| | | AGAGGAATACTAC | |
| SEQ ID | D2_s_323 | TGTAAAACGACGGCC | |
| NO: 139 | | AGTTAGGACTGATAG | |
| | | GGACCAAGATTAC | |
| SEQ ID | D2_as_323 | CAGGAAACAGCTATG | 539 |
| NO: 140 | | ACCATATGTTGACTT | |
| | | ACAGATTCCCCAC | |
| SEQ ID | D2_s_324 | TGTAAAACGACGGCC | |
| NO: 141 | | AGTAAACCAAAGGAA | |
| | | AGAAATAATCGGA | |
| SEQ ID | D2_as_324 | CAGGAAACAGCTATG | 543 |
| NO: 142 | | ACCCCCTCTTATCAA | |
| | | GCTTTCATCACC | |
| SEQ ID | D2_s_325 | TGTAAAACGACGGCC | |
| NO: 143 | | AGTTGATAGGATGTG | |
| | | TCAATGTTCAGTAT | |
| SEQ ID | D2_as_325 | CAGGAAACAGCTATG | 535 |
| NO: 144 | | ACCTTTTGAAACAGA | |
| | | GAGGACTTGGC | |
| SEQ ID | D2_s_326 | TGTAAAACGACGGCC | |
| NO: 145 | | AGTGGACAAAACAAT | |
| | | TTATAGGAAGCCA | |
| SEQ ID | D2_as_326 | CAGGAAACAGCTATG | 402 |
| NO: 146 | | ACCTTAAGAGGAAAA | |
| | | GAAGTTGAGGCAG | |
| SEQ ID | D2_s_327 | TGTAAAACGACGGCC | |
| NO: 147 | | AGTGGAATTCACCCT | |
| | | AAATCTCAACTGA | |
| SEQ ID | D2_as_327 | CAGGAAACAGCTATG | 539 |
| NO: 148 | | ACCACTCTTTCTAAT | |
| | | CTGCCCAATGATG | |
| SEQ ID | D2_s_328 | TGTAAAACGACGGCC | |
| NO: 149 | | AGTAGATCCCAGTAC | |
| | | ATATCCTTTACGT | |
| SEQ ID | D2_as_328 | CAGGAAACAGCTATG | 634 |
| NO: 150 | | ACCTGTAAAGGAAAA | |
| | | TGGCAGGTTACAT | |
| SEQ ID | D2_s_329 | TGTAAAACGACGGCC | |
| NO: 151 | | AGTTCTCACTACCTC | |
| | | TAATCAGCTTCTT | |
| SEQ ID | D2_as_329 | CAGGAAACAGCTATG | 552 |
| NO: 152 | | ACCTCCTAGTACTCT | |
| | | CCCAGGATAGC | |
| SEQ ID | D2_s_330 | TGTAAAACGACGGCC | |
| NO: 153 | | AGTCTTTTGAGCGAA | |
| | | TGGTTTTATGTGT | |
| SEQ ID | D2_as_330 | CAGGAAACAGCTATG | 534 |
| NO: 154 | | ACCTGCATACTTACT | |
| | | GCTTGACTACATC | |
| SEQ ID | D2_s_331 | TGTAAAACGACGGCC | |
| NO: 155 | | AGTCCAATTAAAACT | |
| | | CATGCCCCTTAG | |
| SEQ ID | D2_as_331 | CAGGAAACAGCTATG | 500 |
| NO: 156 | | ACCCTTGAATTCTAT | |
| | | AGGTTTGTGCTGG | |
| SEQ ID | D2_s_332 | TGTAAAACGACGGCC | |
| NO: 157 | | AGTCTAAGGGACATG | |
| | | ACAGAGGTTCT | |
| SEQ ID | D2_as_332 | CAGGAAACAGCTATG | 409 |
| NO: 158 | | ACCCTTACGGTGTCC | |
| | | CTAGCATTTTAG | |
| SEQ ID | D2_s_333 | TGTAAAACGACGGCC | |
| NO: 159 | | AGTGTCCTTTACCTA | |
| | | TGAAGTGCTAGG | |
| SEQ ID | D2_as_333 | CAGGAAACAGCTATG | 479 |
| NO: 160 | | ACCCACCATGCCCTA | |
| | | GTGTCTAATTT | |
| SEQ ID | D2_s_334 | TGTAAAACGACGGCC | |
| NO: 161 | | AGTACGTTTTCACCT | |
| | | GCCATTTAATTG | |
| SEQ ID | D2_as_334 | CAGGAAACAGCTATG | 440 |
| NO: 162 | | ACCTGTCTTCTCTCT | |
| | | ACAAGGACCTCA | |
| SEQ ID | D2_s_335 | TGTAAAACGACGGCC | |
| NO: 163 | | AGTGAAAAGGAAAGG | |
| | | TAGGTAGGTGTTG | |
| SEQ ID | D2_as_335 | CAGGAAACAGCTATG | 464 |
| NO: 164 | | ACCGGACAAGACGTA | |
| | | TAAGACTACTCCT | |
| SEQ ID | D2_s_336 | TGTAAAACGACGGCC | |
| NO: 165 | | AGTGTTGATGTACAC | |
| | | TGGAGAAAGGG | |
| SEQ ID | D2_as_336 | CAGGAAACAGCTATG | 447 |
| NO: 166 | | ACCGTTAAGAAGGCA | |
| | | GGCAATGAGAC | |
| SEQ ID | D2_s_337 | TGTAAAACGACGGCC | |
| NO: 167 | | AGTGAATTTATCTAG | |
| | | GACCCCGTGC | |
| SEQ ID | D2_as_337 | CAGGAAACAGCTATG | 485 |
| NO: 168 | | ACCATCTGACAGGAG | |
| | | TTACAAGAAAGGA | |
| SEQ ID | D2_s_338 | TGTAAAACGACGGCC | |
| NO: 169 | | AGTCTCAGCCTCTCA | |
| | | TTTCTAAGGTTTA | |
| SEQ ID | D2_as_338 | CAGGAAACAGCTATG | 412 |
| NO: 170 | | ACCTTCCTCTCTAGT | |
| | | TACCTTCTTCTCC | |
| SEQ ID | D2_s_339 | TGTAAAACGACGGCC | |
| NO: 171 | | AGTTGTTGTGGATTC | |
| | | CTAGAAATGAAGG | |
| SEQ ID | D2_as_339 | CAGGAAACAGCTATG | 462 |
| NO: 172 | | ACCCTACCCTAGCAC | |
| | | AGATGGATGC | |
| SEQ ID | D2_s_340 | TGTAAAACGACGGCC | |
| NO: 173 | | AGTCTAGGCAAACAC | |
| | | AGTTACTCCTC | |
| SEQ ID | D2_as_340 | CAGGAAACAGCTATG | 450 |
| NO: 174 | | ACCACTCTTTACCAG | |
| | | ATCCAGTATAGGC | |
| SEQ ID | D2_s_341 | TGTAAAACGACGGCC | |
| NO: 175 | | AGTGAGCCTGAGTTC | |
| | | TCATTTGCCTAT | |
| SEQ ID | D2_as_341 | CAGGAAACAGCTATG | 371 |
| NO: 176 | | ACCCATGATTCTTTT | |
| | | ATCTCTCCTGGGG | |
| SEQ ID | D2_s_342 | TGTAAAACGACGGCC | |
| NO: 177 | | AGTCTATCCCTGTTT | |
| | | CAACTCTTTCCAG | |
| SEQ ID | D2_as_342 | CAGGAAACAGCTATG | 555 |
| NO: 178 | | ACCCCACACATTATT | |
| | | CTACCCTGCTG | |
| SEQ ID | D2_s_343 | TGTAAAACGACGGCC | |
| NO: 179 | | AGTCATTCTATGTGT | |
| | | CCAGTCTTAGGC | |
| SEQ ID | D2_as_343 | CAGGAAACAGCTATG | 553 |
| NO: 180 | | ACCGGTGTCAAACTA | |
| | | GAAGATGTGTAGG | |
| SEQ ID | D2_s_344 | TGTAAAACGACGGCC | |
| NO: 181 | | AGTGAGACCTATGAT | |
| | | AAGTGAGCTCTCT | |
| SEQ ID | D2_as_344 | CAGGAAACAGCTATG | 657 |
| NO: 182 | | ACCTAGCCTCAGTGT | |
| | | TTATATCCCACA | |
| SEQ ID | D2_s_345 | TGTAAAACGACGGCC | |
| NO: 183 | | AGTCTAGAGATACCC | |
| | | ATTCTGAACCTCT | |
| SEQ ID | D2_as_345 | CAGGAAACAGCTATG | 551 |
| NO: 184 | | ACCGAAAATGCGTTG | |
| | | AAGTTGAGAGT | |
| SEQ ID | D2_s_346 | TGTAAAACGACGGCC | |
| NO: 185 | | AGTGCCTAATTCTTC | |
| | | TAACACTCTGCT | |
| SEQ ID | D2_as_346 | CAGGAAACAGCTATG | 480 |
| NO: 186 | | ACCTGGGACACACAC | |
| | | TATCATAGACTC | |
| SEQ ID | D2_s_347 | TGTAAAACGACGGCC | |
| NO: 187 | | AGTCCACCTCTATAT | |
| | | TCAGTCAAAGGAG | |
| SEQ ID | D2_as_347 | CAGGAAACAGCTATG | 533 |
| NO: 188 | | ACCAGGGACATAGAC | |
| | | TGGAAGTGGC | |
| SEQ ID | D2_s_348 | TGTAAAACGACGGCC | |
| NO: 189 | | AGTTAAGTTCTGGAA | |
| | | AAGGTCTGATTGC | |
| SEQ ID | D2_as_348 | CAGGAAACAGCTATG | 546 |
| NO: 190 | | ACCGGGTGAATCTGA | |
| | | GTGTCTTAGGT | |
| SEQ ID | D2_s_349 | TGTAAAACGACGGCC | |
| NO: 191 | | AGTAGGAAGTAGGTT | |
| | | AGGGAATGGTAAG | |
| SEQ ID | D2_as_349 | CAGGAAACAGCTATG | 355 |
| NO: 192 | | ACCCAGTGACATCAG | |
| | | AGAATCAAAGGAG | |
| SEQ ID | D2_s_350 | TGTAAAACGACGGCC | |
| NO: 193 | | AGTAGAACATCTCTC | |
| | | TGGCCTAAACT | |
| SEQ ID | D2_as_350 | CAGGAAACAGCTATG | 424 |
| NO: 194 | | ACCGTTTAACCATCT | |
| | | CTTTCCCTGAAGT | |
| SEQ ID | D2_s_351 | TGTAAAACGACGGCC | |
| NO: 195 | | AGTTTACCATCTGAC | |
| | | CTCAAACAATGTT | |
| SEQ ID | D2_as_351 | CAGGAAACAGCTATG | 480 |
| NO: 196 | | ACCCAATCTATGCCT | |
| | | GCAGTGGAATC | |
| SEQ ID | D2_s_352 | TGTAAAACGACGGCC | |
| NO: 197 | | AGTGCCTTCTTTTAT | |
| | | TCATGCGTTGTTT | |
| SEQ ID | D2_as_352 | CAGGAAACAGCTATG | 531 |
| NO: 198 | | ACCCCTTCTCTTCTT | |
| | | GTTTCCTGGTAA | |

1. Method for the detection of chromosome or DNA regions and for the detection of chromosome aberrations, including using directly or indirectly labeled nucleic acid fragments (DNA probes) which are produced with a method comprising the steps:
a) Selecting a number of nucleic acid sequences which occur once in a longer genome section by comparing sequences;
b) Synthesizing sense and antisense primers for a polymerase chain reaction on the number of the selected nucleic acid sequences, where each primer has a sequence which is complementary to the strand or complementary strand of the selected nucleic acid sequence, and also has at least one uniform oligonucleotide sequence which does not hybridize with a sequence of the genome under stringent conditions;
c) Carrying out polymerase chain reactions with the number of sense and antisense primers on the genome section and obtaining synthesized nucleic acid fragments which contain known genome sequences which occur once;
d) Carrying out a second polymerase chain reaction on the synthesized nucleic acid fragments of step c) using a number of primers which hybridize with the uniform oligonucleotide sequence of the primers and obtaining amplified genomic nucleic acid fragments which occur once and which can be used for chromogenic or fluorescence in-situ hybridization of chromosomes with a reduced background.

2. The method according to claim 1, wherein a number of the synthesized nucleic acid fragments obtained after the polymerase chain reaction in step (c) are combined before the second polymerase chain reaction, preferably before their purification.

3. The method according to claim 1 or 2, wherein in step (d) the nucleic acid fragments are labeled or activated by the use of modified or labeled nucleotides, fluorescent or chromogenically labeled nucleotides (NTPs) and dNTPs, hapten-labeled nucleotides, chemically active nucleotides, aminoallyl nucleotides.

4. The method according to claim 1 or 2, wherein the genomic nucleic acid fragments obtained after the first polymerase chain reaction in step (c) are cloned into plasmids.

5. The method according to one of the claims 1 to 4, wherein after step d) a reaction follows to insert reporter groups into the genomic nucleic acid fragments, selected from nick translation, chemical reaction, immunological reaction.

6. The method according to one of the claims 1 to 5, wherein in step (a) the genomic nucleic acid sequences are selected such that products of similar size are produced.

7. The method according to one of the claims 1 to 6, wherein the genomic nucleic acid sequences selected in step (a) have between 100 and 1,000 base pairs, most preferably between 400 and 600 base pairs.

8. The method according to one of the claims 1 to 5, wherein the genomic nucleic acid sequences selected in step (a) are adjacent to each other on the genome.

9. The method according to one of the claims 1 to 8, wherein probes with different labels are produced for the in-situ hybridization.

10. The method according to one of the claims 1 to 9, wherein the genomic nucleic acid sequences selected in step (a) are adjacent to a breakpoint region of the chromosome or flank this region.

11. The method according to one of the claims 1 to 10, wherein the labels of the DNA probes are selected such that they create a fusion signal or a modified fusion signal in the in-situ hybridization of chromosomes.

12. The method according to one of the claims 1 to 11, wherein the label is selected from the group of chromogenic molecules, polymethine dyes, thiazole and oxazole dyes, Hoechst 33342 (2′-(4-ethoxyphenyl)-5-(4-methyl-1-piperazinyl)-2,5′-bi-1H-benzimidazole trihydrochloride), 4′,6-diamidin-2-phenylindole, Alexa 405, Alexa 488, Alexa 594, Alexa 633; Texas Red, rhodamine; sulfonated and non-sulfonated cyanine dyes, Cy2, Cy3, Cy5, Cy7; fluorescent molecules, fluorescein, 5,6-carbofluorescin, FITC (fluorescein isothiocyanate), GFP (green fluorescent protein); chemiluminescent molecules, acridinium; ATTO®-fluorescent dyes (Atto-Tec, Siegen, DE), PromoFluor® dyes (PromoCell GmbH, Heidelberg, DE), MoBiTec® dyes (MoBiTec GmbH, Goettingen, DE), DY® dyes (DYOMICS GmbH, Jena, DE) Quantum Dots; haptens, digoxigenin, biotin, 2,4-dinitrophenol, avidin; enzymes for a chromogenic reaction, peroxidase, horseradish peroxidase, alkaline phosphatase.

13. Probe for an in-situ hybridization for the detection of a chromosome aberration, comprising a number of synthesized PCR fragments, produced using the method according to one of the claims 1 to 12.

14. Probe mixture or test kit which contains several differently labeled probes in accordance with claim 13 for the detection of a chromosome aberration.
